(19) United States
(12) Patent Application Publication
(10) Pub. No.: US 2024/0026663 A1
(43) Pub. Date: Jan. 25, 2024

(54) Handwashing Apparatus

(71) Applicant: Inigo Ackland, London (GB)

(72) Inventor: Inigo Ackland, London (GB)

(21) Appl. No.: 18/248,414

(22) PCT Filed: Oct. 11, 2021
(86) PCT No.: PCT/GB2021/052627

§ 371 Date: Apr. 10, 2023

(30) Foreign Application Priority Data
Oct. 9, 2020 (GB) 2016082.6

(51) Int. Cl. E03C 1/046; E03C 1/05; B08B 3/02; B08B 13/00

(52) U.S. Cl. CPC E03C 1/046; E03C 1/057; B08B 3/022; B08B 13/00; B08B 2203/0217; B08B 2203/0264

(57) ABSTRACT

The present system can also overcome the inability of current automatic hand washing machines to strategically target the hands according to the user's specific needs, and allow the user to control functions, such as the pressure or speed, and duration of water flow, in a touch-free manner. The phrase ‘strategically target’, in the present specification, refers to the ability of the device to automatically detect the location of the hands and actively direct the spray at wherever the user's hands are located within the device's cavity, whilst actively avoiding spraying those areas within the cavity where the user's hands are not located. This targeted spraying occurs with both the water spray and the plain liquid soap spray, even as the hands move freely within the cavity.

### CROSS-REFERENCE TO RELATED APPLICATIONS

This Non-Provisional US patent application claims the benefit of and priority to PCT Application Serial No. PCT/GB2021/052627, filed Oct. 11, 2021, entitled “Handwashing Apparatus,” which claims the benefit of and priority to United Kingdom Patent Application Serial No. 2016082.6, filed Oct. 9, 2020, entitled “Handwashing Apparatus,” the entire contents of both applications of which are hereby incorporated herein by reference.

### BACKGROUND

The present specification relates to handwashing apparatus, particularly apparatus for washing and cleaning a user's hands.

A traditional tap design requires a user to turn or manipulate the tap or other control surface to start and stop the flow of water. The tap or control surface may be dirty or contaminated, which negates the hand washing just undertaken.

To reduce cross-contamination, automatic faucets and automatic soap dispensers have been widely introduced. With or without automatic dispensers, the use of a faucet and sink for washing hands with soap and water is inherently problematic. Hand washing is still usually poorly performed resulting in inadequate removal of harmful pathogens from the hands' surface and the associated health problems that cause unnecessary increase in sickness related absences, hospitalisations and deaths.

One reason for the poor efficiency of washing hands when using a sink and faucet, is the very specific and multi-stage nature of recommended hand washing guidelines, which are often incorrectly followed. The CDC, FDA and WHO all promote the same guidelines, recommending ‘washing hands with plain soap and water’ as ‘the best way to prevent the spread of infections and decrease the risk of getting sick’. Their recommended steps involve a set sequence of wetting the hands with water, soap application, rubbing hands together sufficiently, rinsing off with water, and drying the hands.

A major challenge when using a sink and faucet, in view of the variability of human behaviour, is the lack of consistency in the performance of these steps, with the majority of people adopting ineffective hand washing habits and performing hand washing inadequately.

There are numerous ways in which people do not wash their hands correctly when using a faucet and sink, with people often unaware that they are not washing their hands correctly.

The soap application stage is often missed out altogether. Globally, studies have shown that only 19% of people use soap after defecating: (https://www.wateraid.org/au/articles/4-out-of-5-people-worldwide-do-not-wash-their-hands-after-going-to-the-toilet).

When soap is used, the pre-soap wetting stage is often missed out. This makes the soap harder to spread over the surface of the hands and harder to lather (to encourage rubbing). The tendency is then for the person to activate the faucet's water release to help spread the soap across the hands. Since this action prematurely washes the soap off the hands it naturally prevents the effective performance of the hand rubbing process that is necessary to ensure the removal of harmful pathogens.

Full hand coverage with soap is important to ensure effective handwashing is achieved. Soap molecules contain both hydrophobic ends (which attach to oils, pathogens and other debris) and hydrophilic ends (which attach to water). Due to its bonding with pathogens, the soap enables the pathogens to be rinsed off the hands with water during the subsequent rinsing stage.

Even when soap is used to wash hands it is often used inefficiently so as not to cover the full hand surface, or not enough is applied to enable the effective removal of pathogens from the hands. Many automatic dispensers only release 0.4 ml of soap per activation (in an effort to reduce costs), but this is insufficient to ensure full effective hand coverage, and thus pathogen removal. Since automatic soap dispensers release a pre-set volume of soap onto a hand, and hand sizes vary greatly, a common uncertainty amongst people when washing hands is whether they have applied enough soap on their hands, particularly if they have large hands. Problematically, a typical user has no way of gauging whether a single release of soap is sufficient to fully cover and clean their hands effectively, given that the soap lands on a small region (typically the palm) of one of the hands. To compensate for this, people often activate an automatic soap dispenser multiple times, and in doing so waste excessive soap. People also often apply excessive amounts of soap when to the initial wetting stage has been omitted due to the increased difficulty in spreading soap without prior water application.

Another problem with faucets explicitly relates to poor hand rubbing practices. Hand rubbing is an essential part of the handwashing process, and the longer the hands are rubbed together with soap on them, the greater the number of pathogens that are dislodged and able to be removed from the hands with the subsequent water application. Repeated studies have revealed that this stage is inadequately performed by most people.

Part of the problem with faucets, with regards to effective handwashing, is the ability of a user to rapidly activate water release by simply placing a hand beneath a faucet, and since handwashing is performed multiple times a day there is a significant temptation for a user to activate the rinsing stage early and cut short both the spreading of soap across the hands and the rubbing of the hands together.

There is nothing inherent within a faucet's logic that ensures the correct hand washing sequence is consistently performed and in an effective manner. Whilst taps have been used for many centuries and are universally associated with washing hands, they have many inherent problems that negatively impact upon the quality of the hand wash performed.

By virtue of their protrusion into the space above a sink, and the (physical) space they occupy, faucets physically interrupt the free-flow of hand movement in the area above a sink, which disrupts the flow of the handwashing experience. Faucets not only pose a visual obstruction to the direct line of vision between a user and parts of their hands during the hand washing process, (particularly during the hand rinsing stage, and if the hands are rubbed together beneath the faucet) but also pose a visual obstruction and distraction to any information relating to hand washing that could be placed on the surface behind where the faucet is situated which would otherwise be an ideal place for information relating to handwashing to be located.

By their very presence, whether manually operated or sensor activated, faucets pose an added cross-contamination risk during hand washing (whether through accidental or intentional hand contact). This is particularly pertinent during the hand water application stages when the faucet is turned on or off and when hands are placed beneath the faucet to catch the falling water, due to the relatively close proximity of a user's hands to the faucet. Furthermore, given that water is released by a faucet from above the sink, and users often move their hands above the basin to receive the water or rub the soap in, surfaces near to the faucet, including the surface the faucet is mounted upon, are at high risk of contamination, whether from direct user contact, or from the dripping or spraying of waste water from the user's hands. If a person is looking at information located in front of them whilst washing their hands, they have a much higher chance of making accidental physical contact with the faucet, creating a cross-contamination risk.

When water is released from a faucet located below a user's eye level their hands will be partially obscured from view by that faucet whenever the hands are placed beneath it to catch the falling fluid, increasing the likelihood of unintentionally touching the faucet. The risk of cross-contamination posed by inadvertent contact with a faucet is particularly pronounced for those who are visually impaired or have other disabilities such as movement disorders.

The same problem and contamination risk arises when activating soap release, whether from a faucet or a soap dispenser. Given that most soap dispensers contain a fairly limited volume of soap, these frequently run out; when soap release is meant to be activated by an IR proximity sensor, but the soap dispenser fails to dispense any soap, it is a natural instinct for many users to place their dominant hand progressively closer to the soap outlet (which is located above their hands, but beneath their eye level) increasing the risk of direct physical contact with the soap outlet, and subsequent cross-contamination.

Since hands need to navigate beneath the correct outlet point in order to receive the water or soap, the consequent hand movements required to fulfil effective hand washing in the correct sequence creates a fractured hand washing process, and encourages stages to be missed out. The further a hand needs to travel between different outlets, the greater the disruption and the more likely a user is to skip parts of the hand washing process. If the outlets are sensor activated and closely located, the more likely it is for the wrong outlet to be activated and the greater the scope for user uncertainty (particularly if any direct view of the outlet is obscured from the user by the faucet).

Due to the relatively narrow diameter of water flow from a faucet, poor hand positioning or subtle hand movements can result in the water released bypassing the hands and making insufficient contact with the hands. Conversely, if a faucet's water stream were wider than that of the hand it would invariably result in a lot of water being wasted unnecessarily. Water, as with soap, is often wasted by dripping or falling between the gaps in the fingers, is released onto one side of the hand, and the spreading of the water or soap for full hand coverage is dependent on the user, which takes time and willingness.

Most faucets release water close to the rear of the sink and sinks are relatively shallow, meaning user's hands are often naturally in close proximity to a sink's rear wall, the faucet outlet and the base of the sink when receiving water, posing a contamination risk from multiple surfaces.

Three major problems with the use of a sink and faucet for hand washing (regardless of whether automatic sensors are used), are (i) inefficient hand washing including both the sequence of the wash, and the quality of the wash performed, (ii) water and soap waste, and (iii) high risks of cross contamination.

There have been a few attempts to provide automated hand washing devices, but these have shown limitations, notably relying on the inclusion of antimicrobials, the inability to comply with the CDC guidelines for effective hand washing (including with respect to the hand rubbing stage), the use of fluid that has recurrent contact between the hands and the container, the inability to sufficiently rinse off chemicals or heavy debris (which is of particular significance with food handlers, healthcare workers or those working on the land), a contamination risk associated with the narrow diameter of the separate hand cavities into which the hands are placed, resource inefficiencies, and the lack of ability to modify a wash according to an individual's needs without disrupting a hand wash process that is underway.

‘Automatic handwashing devices’, in the current specification refers to automated devices within which the user can place their hands to undergo a pre-determined complete hand hygiene process (from within the device) as opposed to a simple activation of an IR proximity sensor beneath a faucet or dispenser that automatically dispenses a solution when activated.

Automated hand cleaning devices rely on antimicrobial sanitisers or solutions to kill bacteria, and are typified by single-hand cavities where hands are unable to be rubbed together to dislodge pathogens and be rinsed away.

Overuse of antimicrobials poses the potential risk of microbial resistance emerging. With regards to hand sanitisers, organisms particularly sensitive to becoming resistant to antimicrobials are gram negative bacteria such as E. coli, and mycobacteria which can lead to gastro-intestinal infections such as diarrhoea and tuberculosis. These bacteria could pose serious health problems if the resistant bacteria become prevalent in the community.

By contrast plain soap poses no such risk. Accordingly plain soap and water are recommended by all leading health organisations globally for hand washing use in the general population. The FDA and CDC both advocate against the general use of antimicrobial soap because ‘studies have shown that using antibacterial soap may contribute to antibiotic resistance’ (https://www.cdc.gov/handwashing/faqs.html).

According to the FDA “There is currently no evidence that consumer antiseptic wash products (also known as antibacterial soaps) are any more effective at preventing illness than washing with plain soap and water. In fact, some data suggests that antibacterial ingredients could do more harm than good in the long-term . . . . ” (https://www.fda.gov/drugs/information-drug-class/qa-consumers-hand-sanitizers-and-covid-19)

Whilst automatic hand hygiene devices are known, these suffer several drawbacks which limit their widespread use.

Due to the use of single-hand cavities for each hand, the hands in automated hand hygiene devices are unable to be rubbed together within the device to dislodge pathogens (as recommended by the CDC's hand washing guidelines), that would then be rinsed away with water. Furthermore, the fluids in each narrow cavity also make frequent contact with both the hand and the cavity's sides meaning that they rely on antimicrobial sanitisers or solutions, as these kill bacteria. Even though antimicrobial sanitising devices use significant resources, the logic for having smaller cavities is to reduce the overall amount of sanitising fluid required with any sequence activation.

Current automated sanitisation systems cannot ensure that heavy debris will be removed from the hands. They are also not suitable for hand washing when the hands have been in contact with chemicals or have heavy debris on them due to the fluid in the cavity making repetitive contact with both the hand and the container walls, (which would result in chemicals or debris having prolonged hand contact). Some known automated hand hygiene stations, where there is heavy debris that needs removing, require the additional use of a faucet before undertaking the wash using antimicrobials.

When compared with the amount of fluid released by standard IR proximity activated water faucets and soap dispensers, automated sanitisation systems that use antimicrobials are very resource intensive, given that they function on the logic of covering everywhere with antimicrobial spray or solution, regardless of a user's hand size.

Whilst small and separate containers for hands reduces the scope of resource inefficiency and waste compared with larger hand containers, the relatively narrow openings of single-hand cavities pose an increased risk of becoming contaminated when dirty hands enter them, and of acting as a source of recontamination as the hands are withdrawn; people with visual impairments or a physical disability that affects motor control are at particularly high risk of this cross-contamination. Relying on antimicrobials during the washing process does not exclude the risk of recontamination from the cavity opening itself, but does reduce risks of cross-contamination from circulating or spraying fluids that repeatedly contact or splash both the hands and the cavity walls when the hands are placed within the narrow cavity chambers.

In order to operate automatically, automated washing systems typically have a set washing operation, which may not be suitable for every situation.

For instance, if someone has only lightly soiled hands, for example if they have spilt fruit juice on them, they may require only a rapid rinse. However, an automatic system will generally execute the full program, leading to unnecessary washing duration or operations; as a result, the hand washing process is inefficient, and people are more likely to avoid washing their hands. The resulting washing operation also unnecessarily wastes water and soap.

There is though a need to encourage people to adopt sufficient hand washing.

Conversely, if someone has heavily soiled hands or has been in contact with potentially hazardous material (whether chemical or potential exposure to pathogens), the automatic antimicrobial washing (or sanitisation) cycle may not be sufficient, leading to ineffective hand hygiene, or the necessity to repeat the process.

A further problem is people who do not engage in proper hand washing technique, whether this be the amount of soap used, or the time spent washing, the rubbing of the hands together with the proper technique, or other factors.

There have been a number of attempts to improve hand washing efficacy by monitoring of users and recording their behaviour (often involving individual identification and surveillance, to try to pressurise compliance), by increasing awareness of proper techniques and process through posters and information dissemination campaigns of the correct hand washing procedure and through using anti-microbial alternatives to soap and water.

Current methods of trying to ensure efficient hand washing, have proved ineffective, given the relatively low adherence to proper hand washing techniques. According to a 2018 study from the U.S. Department of Agriculture, ‘when it comes to handwashing before meals, consumers are failing to properly clean their hands 97 percent of the time’: https://www.usda.gov/media/press-release/2018/06/28/study-shows-most-people-are-spreading-dangerous-backeria-around

There is accordingly a need for a handwashing device that overcomes many of the limitations in the prior art, particularly in relation to ensuring official handwashing guidelines are consistently adhered to, the hand washing is carried out in a more time and resource efficient manner, the inefficiencies caused by human behavioural variability are minimised, and the risks of cross-contamination are reduced.

Neither automated devices nor sinks and faucets have yet produced a satisfactory solution that overcomes the many challenges that currently exist in relation to ensuring effective hand washing is consistently achieved.

### SUMMARY

A key object of the present invention is to ensure a more efficient and effective automated hand washing process that automatically ensures the consistent fulfilment of hand washing in accordance with the CDC's recommended hand washing guidelines, and does so without use of antimicrobial spray or solution (i.e. it uses plain soap and water).

Another object of the invention is to enable the CDC's recommended hand washing guidelines to be completed in a fully touch-free and more time-efficient manner, where there is minimal disruption between the steps of the varying stages of the handwash so as to ensure as seamless a flow as possible (in the sequence of separate water and soap applications, and hand rubbing and rinsing), as well as to reduce the potential for human error, confusion or uncertainty during the washing process.

In light of the extreme water poverty, which affects a quarter of the world's population, the universal necessity for frequent hand washing and the current high waste of resources (water and soap) during the hand wash process, the invention aims to consistently provide a more resource efficient way of washing hands.

A further objective of this invention is to reduce risks of cross-contamination

A further object of the present invention is to provide a more convenient hand washing system which encourages users to wash their hands, and to wash them in an effective and proportionate manner. Another separate object is to allow users to customise the washing process for their needs, or to have this done automatically without disrupting the sequence of the handwash. Other separate advantages, which may be independently utilised, will become apparent from the description and drawings.

According to the present invention, there is provided a handwashing device according to independent claim 1 or any of the points of interest A0001 to A0014.

The present system can also overcome the inability of current automatic hand washing machines to strategically target the hands according to the user's specific needs, and allow the user to control functions, such as the pressure or speed, and duration of water flow, in a touch-free manner.

The phrase ‘strategically target’, in the present specification, refers to the ability of the device to automatically detect the location of the hands (wherever they are located within the cavity) and (actively) direct the spray at wherever the user's hands are located within the device's cavity, (whilst actively avoiding spraying those areas within the cavity where the user's hands are not located). This targeted spraying occurs with both the water spray and the plain liquid soap spray, even as the hands move freely within the cavity.

Ultimately, this ensures more effective hand washing. It also reduces water and other resource wastage.

The handwashing system discussed herein is described principally as involving the separate application of water, and plain liquid soap (which may of course also include water) on the other; however, it will be appreciated that a single cleaning fluid, or separate applications of different cleaning fluids could equally be used, and these could include solvent based cleaning fluids, depending on the application.

The handwashing system discussed herein is particularly directed to deploying soap and water as separate fluids. Some known systems dispense a single antimicrobial solution, however deploying water and soap solution separately is more effective, particularly for removing heavy debris.

The provision of a relatively large, open cavity which can accommodate both hands allows the user to rub the hands together; this friction is particular effective for cleaning hands.

The handwashing system discussed herein obviates the need for a faucet; the use of precision targeted nozzles provides many advantages, which will be discussed herein.

It should also be noted that deploying soap and water only, and allowing hand rubbing, is compliant with current CDC guidelines on hand washing.

The present system carries out cleaning steps automatically and in sequence, making the cleaning process convenient and efficient, and ensures that cleaning steps are not missed by the user.

By handwashing we are referring to any process or system that enables a user to carry out the correct procedure for effective handwashing, as recommended by the CDC, FDA, and

WHO guidelines, which all recommend following the same five handwashing steps (of wetting the hands with water, soap application, rubbing hands together, rinsing off with water, drying hands), and which stress the importance of following the correct hand rubbing process in order to optimise the removal of pathogens from the entire surface of hands, when washing hands.

According to the CDC, FDA, and WHO guidelines, washing hands with plain soap and water is the best way to get rid of germs in most situations.

The objective of the present device is to provide an automated means of ensuring hands are washed efficiently and effectively whilst adhering to the guidelines recommended by the CDC, FDA and WHO for effective hand washing. The present device is a fully automated, smart, touch-free handwashing system that uses ‘plain’ soap (i.e. without antimicrobial agents) and water and facilitates effective hand rubbing (within its cavity) in accordance with official recommendations. Furthermore, the present device ensures the correct sequential order for effective hand washing is achieved. The present device does not possess the limitations of requiring antimicrobials, or of requiring the use of a faucet, to operate effectively. It also does not require the user to place their hands beneath an individual outlet for the dispensation of a specific fluid despite its separate applications of both soap and water.

### BRIEF DESCRIPTION OF THE DRAWINGS

The invention will now be described, by way of example, with reference to the drawings, of which

FIG. 1 is a perspective view of the handwashing device;

FIG. 2 is a section side view of the handwashing device;

FIG. 3 is a perspective view of a detail of the cavity of the handwashing device;

FIG. 4 is a perspective view of a moving unit of the handwashing device;

FIG. 5 is a section side view of the handwashing device showing one of the process steps;

FIG. 6 is a section side view of the handwashing device showing another of the process steps;

FIG. 7 is a sectional elevational view of the handwashing device showing the moving unit on the back wall and the display screen;

FIG. 8 is a schematic diagram of the handwashing device internal systems;

FIG. 9 is a diagrammatic view of the control and logic architecture;

FIGS. 10 and 11 is a flow chart showing steps of one hand washing process (step 560 of FIG. 10 leading to step 561 of FIGS. 11); and

FIGS. 12 and 13 is a flow chart showing steps of an alternative hand washing process (the final step 580 of FIG. 12 being the initial step 580 of FIG. 13).

### DETAILED DESCRIPTION

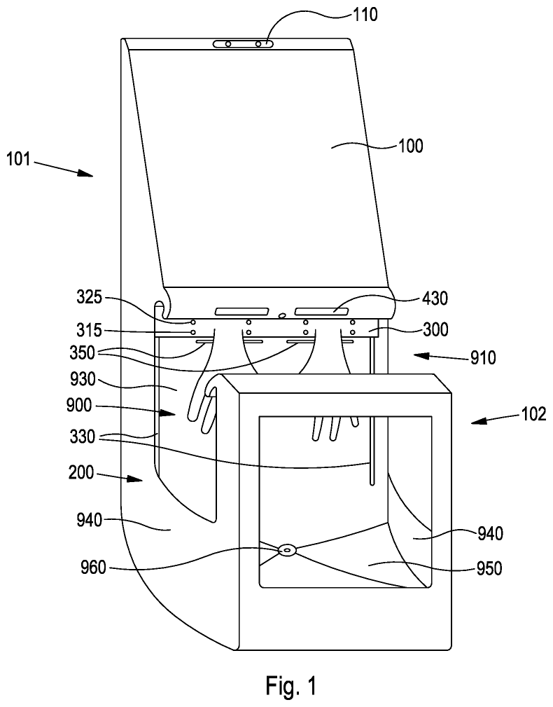

Referring to FIG. 1, a handwashing device 101 comprises a housing 200 and a hand washing portion 102. The hand washing portion 102 comprises a front wall 920, rear wall 930, bottom wall 950 and side walls 940 which together define a chamber or cavity 900 into which a user may insert their hands to be washed and cleaned. The housing 200 includes the control means and handwashing components.

Cavity and Moving Unit

The smart handwashing cavity is characterised by having a single open (and downwardly extending) cavity whose shape is designed to accommodate downwardly extending hands within the cavity and to accommodate the free movement of both of the hands even when rubbing together. The entire washing process, which includes the release and receipt of water, and of soap onto hands, and the rubbing of hands can be efficiently performed from within the cavity as a user's hands are extended down within the cavity.

Nozzles on opposing walls (front and back) simultaneously target the hands that move within the cavity space, obviating the need for a faucet or any liquid dispensed external to the cavity.

Sensors from within the cavity determine the location, orientation and movements of the hands in order to deliver targeted spray.

The cavity facilitates a fully automated hand wash, in accordance with the CDC's recommended sequence using only plain soap and water (not requiring the use of antimicrobials) to ensure the wash's efficacy.

The cavity enables the uninterrupted transition between the various different stages of the hand wash for a more seamless handwashing experience, by targeting the respective fluids dispensed (i.e. soap and water) onto the hands. This means that the hands' movement does not need to be restricted, nor do they need to be placed beneath a specific sensor or outlet, or be consciously manoeuvred beneath a water or soap flow to achieve full hand coverage.

By dispensing its soap and its water onto wherever the hands are located and avoiding the areas where they are not located, with every wash it automatically customises the amount of fluid dispensed according to the specific user's hand characteristics, thereby optimising resources used.

Typically, the height of the insides of the open cavity will be greater than 30 cms to allow for all hand sizes (from the creases of the wrists to the fingertips) to be accommodated when extended down within the cavity as well as allowing sufficient additional space to keep the tips of the fingers sufficiently clear from the base of the cavity, although in general it will be much longer than this to allow for the additional accommodation of extended forearms.

The width will vary according to the number of users intended for the cavity, but for a single user it will typically be approximately 44 cm.

The walls of the sides (connecting the front and rear of the cavity) may be less high than the walls of the front and rear walls, and in some instances may be only a small fraction of their height. In addition, these side walls may be of a transparent material.

The bottom of the cavity (base) will have a concave surface so that any waste fluids collecting at the sides drain freely to a waste outlet.

The depth of the device's cavity (the distance between its front and back wall) will typically be about 26 cm.

The cavity dimensions are sufficient to allow freedom of hand movement and the rubbing of both hands together to occur within the cavity whilst making it easy for hands to avoid touching the sides of the cavity and mitigating risks of hands being contaminated by splashback when receiving water and soap from angled atomising nozzles.

The front and rear walls may have slight concavity in order to enable more efficient spraying and less chances of water splashing occurring.

Antimicrobials are not required because the officially recommended guidelines can be followed efficiently including effective rubbing, and there is less likelihood of cross-contamination during the washing process. This reduced risk is helped by the large, relatively open cavity which can accommodate both hands for effective rubbing within the cavity, as opposed to the narrow single-hand cavities that typify known automatic hand sanitisers where the antimicrobial solution released has repetitive contact between the hands and the container walls.

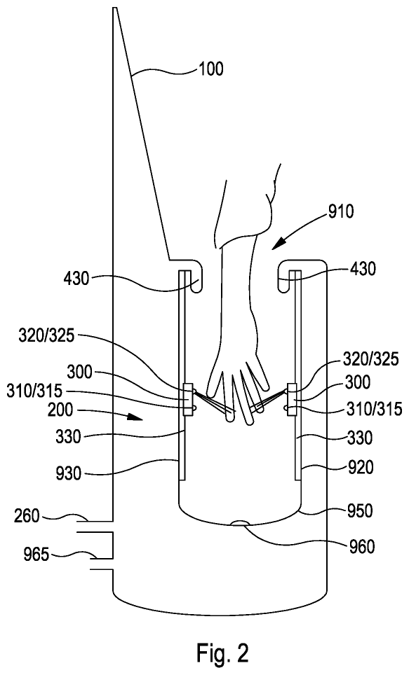

Referring also to FIG. 2, the front wall 920 and rear wall 930 of the cavity 900 are oriented parallel to each other and spaced sufficiently to allow the user to insert their hands between them with room for moving the hands. A bottom wall 950 extends between the front wall 920 and rear wall 930. The bottom wall 950 has a sloping concave surface that catches and directs used water and soap to a waste outlet 960 to be directed into the waste system via the waste outlet pipe 965. Side walls 940 may extend perpendicularly between the front wall 920 and rear wall 930, and upwards from the bottom wall 950, but need not extend the full height of the front wall 920; the side walls 940 prevent water from the washing process from escaping the handwashing device 101, but are chosen to be low enough to allow convenient hand movement and access to the cavity.

The open sides allow greater user visibility of the hands (given the extended depths of the cavity) and greater freedom of hand movement, with less risk of contamination from inadvertent touching of the side walls.

The open sides allow increased air circulation within the cavity thus maintaining a fresher, and less humid environment, which will be less likely to harbour the growth of moulds and pathogens. It is important that the hand washing area, including the cavity's surfaces, is kept as clean as possible, especially since antimicrobials are not being used.

A further benefit of having open sides is that it allows multiple single handwashing devices to be placed side by side thus creating an elongated handwashing device, without large modifications being required.

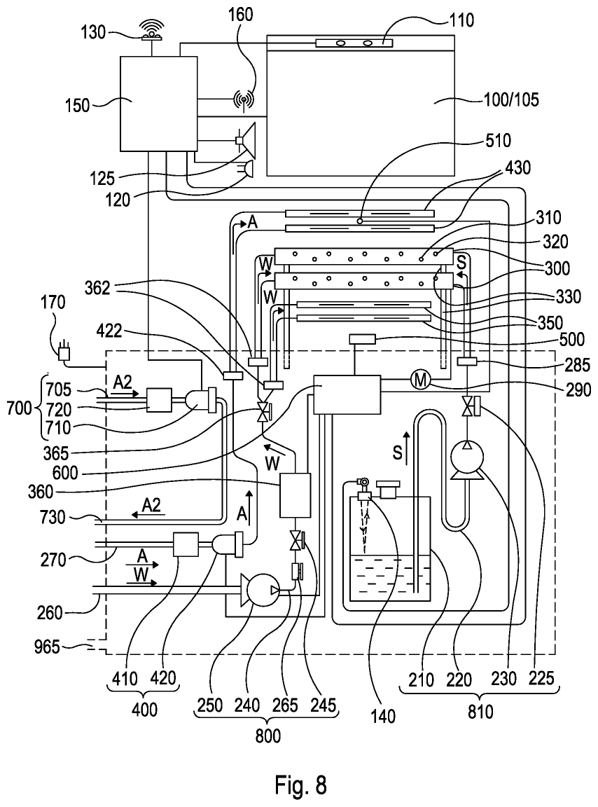

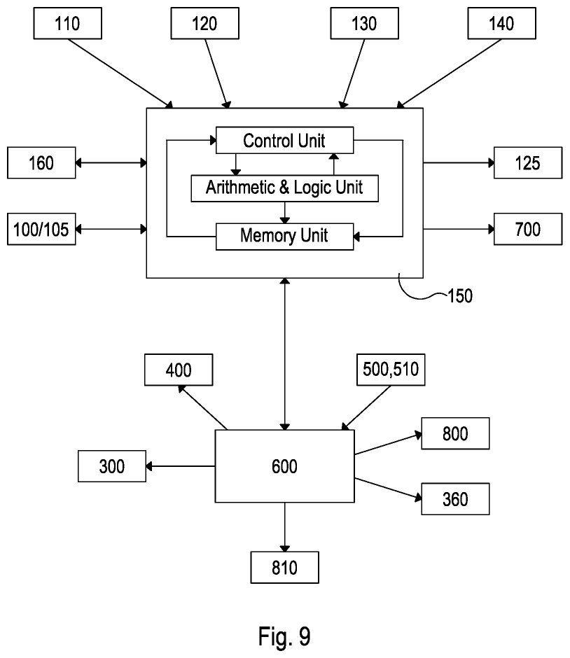

Referring also to FIG. 8, water 310 and soap outlets 320 are provided on two parallel moving units 300, one of which is situated on the rear wall 930 of the inner housing, and the other on the front wall 920 of the inner housing. The water 310 and soap outlets 320 each respectively comprise an array of water atomising nozzles 315 and soap spraying nozzles 325 to direct a spray of water and soap into the cavity 900. A water pipe 240 and soap pipe 220 supply the moving unit 300 with water and soap.

Each separate moving unit consists of a single rectangular bar, through which runs a water pipe that leads to atomising nozzles for dispersion of the water, and a soap pipe that leads to atomising nozzles for dispersion of the soap. The moving units move up and down within the cavity along a vertical axis, much like parallel elevators moving in unison (in contrast to horizontal movement that would be akin to a conveyor belt; if the moving unit was horizontally inclined and sprayed the hands the fluid would fall back onto the lower moving unit, pose a contamination risk and risk clogging the moving units fluid outlets with the dirty water and soap that falls off the hand). The nozzles on each moving unit will move up and down (in a vertical direction) with the moving unit, in sync with the corresponding nozzles on the moving unit directly opposite, in order to be optimally placed to target the water or soap onto specific parts of a hand that is downwardly disposed into the cavity.

References made to ‘soap spraying nozzles’ refer to soap atomising nozzles. Having nozzles strategically placed on opposing sides of the cavity enables the targeted spray to cover both sides of the hands simultaneously. This results in more effective hand washing by speeding up the application of water and soap without skipping or altering the CDC's recommended sequence.

The atomising nozzles allow for a finer and more even coating of soap across the hands, than is achievable with normal applications of soap on the hands' surface during hand washing, particularly given the relatively high viscosity of soap.

This reduces the amount of soap required to achieve full hand coverage and effective hand washing.

When the hands have been inserted into the device, with the fingers pointing down (towards the base of the cavity), the position, orientation and location of the hands are determined. The CPU will activate adjustments to the angle of the appropriate nozzles such that any trajectory of flow from any activated nozzle will reach the hand. The nozzles will be activated to direct the flow of water, and soap at the dorsum and palm of the hand, down along the thumb, and down the fingers. Since the thumb naturally juts out at an approximately 45 degree angle from the main body of the hand, a nozzle targeting the thumb (with fluid) would have to follow along the angle of the thumb rather than moving in the same downward direction as the more vertically positioned fingers. Due to the hands' natural shape, and the potential for large gaps between fingers when the fingers are abducted, non-targeted spray would result in a lot of soap and water waste to achieve full hand surface coverage.

This targeted spray would not be possible if the soap and water were released from a faucet outside the cavity. It also would not be possible using a simple proximity sensor, as is customarily used in relation to handwashing release activations, and would be far more difficult to achieve in a small single-handed cavity given the proximity of the hands to all walls of the cavity.

Air outlets for drying the hands could also be positioned on this moving unit (this is not shown), instead of at the entrance 910 to the inside of the housing of the device. Alternatively, water and soap outlets could be provided from a plurality of nozzles on the front and/or back walls instead.

A motorised pulley or trolley 330 is used to move the moving unit 300 up and down within the housing.

Sensors 500 within the hand cavity 900 continuously detect the location of the hands wherever they are situated within the cavity 900; ideally, the sensors also determine the orientation and movements of the hands in order to deliver targeted spray, and to detect gestures made in the cavity. One or a plurality of sensors may also be located on the moving unit. The sensors detect an object's proximity, and provide an accurate real-time assessment of the area of the hands to be sprayed with soap and/or water. This means that the nozzles can target the actual hand and automatically accommodate the hand size of a user without requiring any delay to the wash process or prior knowledge of the identity of the user.

The fluid (soap and water) dispensing nozzles have the ability, via sensors, to precision-target the hands, wrists and forearms to achieve a hand-glove printing effect. This allows full hand coverage with soap and water which can be achieved without restricting the hand movements but also avoids spraying areas in the cavity where the user's hands are not located which reduces soap and water waste.

The smart precision-targeting (of the nozzles) customises where the fluid is sprayed at each stage of the hand washing process, (by altering features such as the trajectory, angle and volume of fluid dispensed) to ensure that the dispersion area where the fluid is dispensed corresponds with the real-time location, orientation, size and shape of the hands within the cavity space, whilst avoiding spraying areas where the hands are not located within the cavity.

The nozzles which are sited on opposing sides of the cavity walls, release their targeted sprays on both sides of the hands such that both hands are fully covered simultaneously, regardless of where, or how far apart the hands are situated from one another within the cavity, and without spraying areas where those hands are not located.

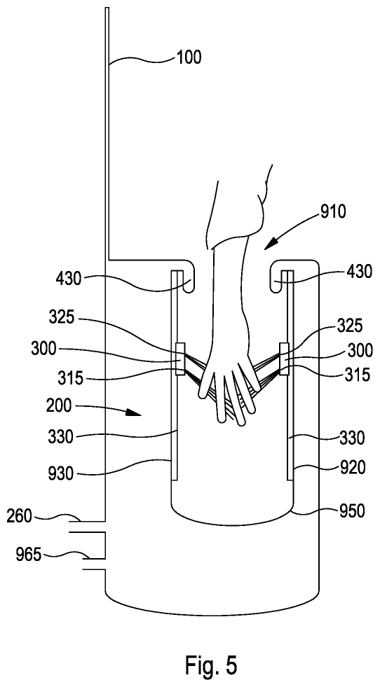

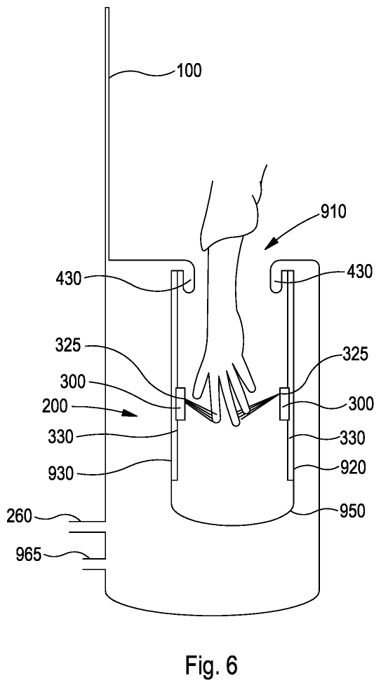

Referring also to FIGS. 5 and 6, to wash a user's hands, the moving unit 300 travels down the trolley 330 while water and soap are dispensed by water atomising nozzles 315 and soap spraying nozzles 325 respectively, particular nozzles being activated corresponding to the location of the user's hands.

The speed of the moving units may be adjustable, together with the volume of soap and water delivered to various parts of the hands or arms, (e.g. if a user has very oily forearms, the moving units may move more slowly down the forearms).

In this manner, the water and soap are dispensed in a targeted manner, using only the nozzles which are adjacent to the user's hands, reducing the amount of water and soap used. This is particularly useful for an elongated multi-user hand washing variant of the system.

Some or all of the water atomising nozzles 315 and soap spraying nozzles 325 could also be directionally alterable, as determined by CPU so that the dispensed water and/or soap is better directed to the user's hands. Soap is more viscous than plain water, however it is possible to deliver atomised soap by using appropriate nozzles and pressure. Slight increases in temperature can also be used to significantly reduce the viscosity of soap to facilitate its spraying.

The detection of real time location (and orientation) of hands in the cavity allows the spray of only those areas where the hand is located, rather than whole cavity volume. Sensor detected information is relayed back to the CPU which determines which nozzles to release spray from and for how long.

The nozzles are ideally atomising nozzles, which allows for a fine spray of soap.

The location of the nozzles on both the front and rear walls of the cavity ensures that both sides of both hands are covered simultaneously, without the need for the user to rotate their hands in order to receive full hand coverage of the soap and the water.

Whether through parallel moving units that directly travel down and up the cavity's vertical axis passing over both sides of the hands, or nozzles that actively angle downwards from the cavity wall, this strategic targeting logic serves to print the soap onto both sides of both hands within sizable cavities in a resource-efficient manner.

When water and soap has been sufficiently dispensed onto the user's hands down to the fingertips, the water and soap flow ceases, and the downward movement of the moving unit 300 stops.

The speed at which the moving unit 300 descends can be varied in order to apply more soap and water to a particular region of the user's hands, and the flow of soap and water can be varied, both to adjust for the number of nozzles being used at a particular time, and to vary the amount of soap and water dispensed through the nozzles.

This arrangement reduces the number of nozzles that the cavity 900 requires to apply soap and water to a user's hands compared to an arrangement of fixed nozzles. The combination of the provision of a cavity which can accommodate both hands, and nozzles which can deploy water and soap in a targeted manner, mean that the system is highly efficient in the use of resources, particularly with regards to the use of soap.

Automatic Start

The Automated sequential order of the hand wash (with no direct user determination required) is facilitated by virtue of the smart, large, open cavity that maps the hands' location within the cavity in real time, fed back to the CPU, and the fact that all of the applications (e.g. soap and water, but in some embodiments including air for hand drying) function within a cavity that is large enough for a user's hands to comfortably extend downwards into it.

Furthermore, the ability to perform gesture control within the cavity allows for the seamless modification of the wash.

The device automatically starts when the hands of a new user are inserted into it, with a fully automated uninterrupted sequence initiated, involving targeting nozzles on parallel walls wetting the hands, then application of soap, followed by an alert for the user to rub their hands. The rinsing stage begins automatically after sufficient time has passed to enable a user to rub their hands together effectively.

The rinsing stage will then automatically stop after a predetermined time, and a dryer is triggered when the user raises their hands towards the cavity entrance. These steps can all be completed without any input from the user apart from inserting and removing their hands from the cavity.

The opportunity still exists for the user to modify the wash, if desired, by very simple actions such as, for example, continuing to rub their hands to extend the rinse stage. This results in a seamless and efficient hand wash that follows the CDC recommended guidelines, whilst still giving the flexibility to modify this wash (for example if the hands have extra debris on them) with minimal effort and without creating a break in the flow of the handwash.

Referring to FIG. 1, the housing 200 contains the water and soap handling means, and the control and processing systems, and the handwashing portion 102 is located on the lower part of the housing 200 accessible by the user from the front. On the upper front part of the housing is mounted a display screen 100, and a computer vision sensor unit 110 conveniently located above it.

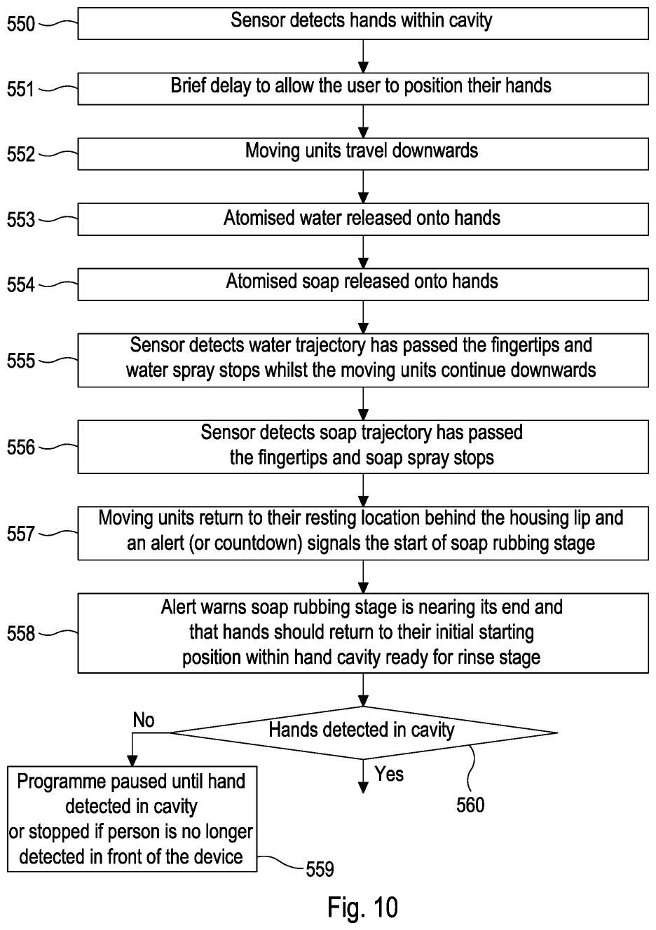

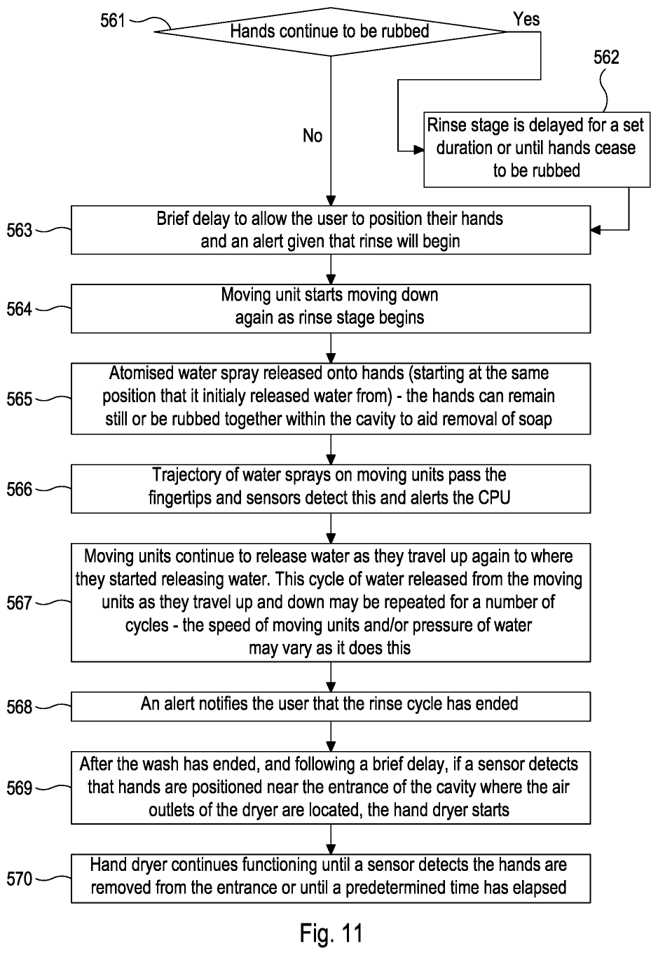

Referring also to FIGS. 10 and 11, the sensor 500 detects the presence of a user's hands within the cavity 900; a delay 551 may be included to allow time for the user to position their hands. The moving units 300 are then automatically lowered 552 to dispense water 553 onto the user's hands, ideally in a directed manner as described above, and then dispense soap 554 onto the user's hands. It will be realised that references to dispensing water and soap could include dispensing these in either order one after the other, or simultaneously.

When the sensors 500 detect 555 that the moving units 300 have reached the fingertips of the user, the water atomising nozzles 315 and soap spraying nozzles 325 stop dispensing soap and water 555, 556. The moving units 300 will then relocate to their starting positions and an alert may sound 557 to indicate to the user that the water and soap dispensing stage has completed, and the hand rubbing stage will commence. Alternatively, the moving units 300 may be raised and further soap and water applied (for a longer soap washing stage), as determined by the control system as described below.

The automated delay between the soap application across the hand's full surface whilst the hands are covered in soap, and the hand rinsing stage, will naturally encourage an efficient rubbing stage. Since the user has hands covered with soap (which is in a latherable state due to the prior application of water), and there is no faucet to tempt the user to prematurely rinse the soap off their hands, meaning that the most natural, and least effortful approach is for the user to rub their hands together during this delay.

The temptation to rinse soap off before it has been properly rubbed into the hands, which is a major obstacle in consistently achieving effective handwashing when faucets are involved, is averted. The hand rubbing can occur within or above the hand cavity. This rubbing stage can be shortened by the user's gesture or prolonged if sensors detect that the user is still rubbing their hands together.

When the hand rubbing stage is completed the user may be alerted with a sound and/or message to notify them to return their hands to the initial starting position 550 within the hand cavity ready for the rinse stage to begin 558. The sensors 500 check 560 whether or not the user's hands are detected in the cavity 900, and if they are not detected within the cavity and the user is no longer detected in front of the device 559, the sequence stops and awaits a new user.

If the user's hands are detected in the cavity, the sensors 500 check to see if the user is still rubbing their hands 561. If the user is still rubbing their hands, the sequence may be delayed to allow the user to finish this 562, 563.

When the rinse stage begins, the moving units 300 start moving downwards, while dispensing water 564, 565 from the water atomising nozzles 315. As before, the water is directed onto the sensed region that the user's hands occupy. When the sensors detect that the moving units 300 have passed the user's fingertips 566, direction of the moving units 300 are reversed and further water is applied to the user's hands as the moving units 300 are raised 567. This sequence may be repeated, dependant on the control unit, as will be described below. When the rinse sequence has completed, the user may be alerted with a sound and/or message.

The user may choose to finish the washing process at this time; the sensors 500 check to see if the user's hands are still present in the cavity 900 and if they are, a drying sequence is initiated. For the drying sequence, filtered air is blown into the cavity 900 (which may be heated), in order to dry the user's hands. When the sensors 500 detect that the user has withdrawn their hands from the cavity 900, the drying sequence is stopped, and the handwashing device 101 is reset to await the next user.

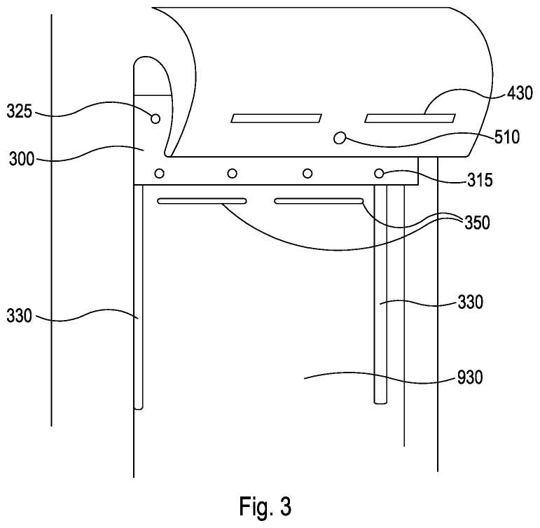

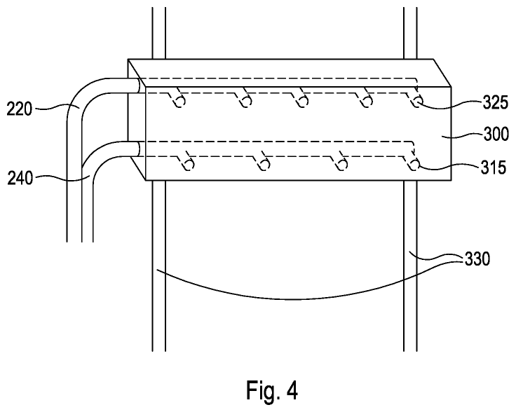

Referring to FIG. 3, additionally one or more water sheet outlets 350 may be provided to produce one or more water blades or sheets to dispense water to the user's hands as they are inserted into the cavity 900.

The addition of a water blade/sheet within the cavity to sheer off extra debris and/or give a more powerful rinse, further enhances the efficiency of the device, and can be used in combination with the atomising nozzles to provide a quicker and more powerful boost to the washing process (if required). This acts as a wide and thin pressurised jet of water that can sheer debris off the hands. Having two parallel water blades/sheets has an additional benefit in that both the front and back of the forearms, wrists, or hands can have debris rapidly removed at the same time, and with reduced hand movement required. It can also avoid the need to rub the debris off directly with the hands, which often involves spreading the debris further over the hands before it is removed.

The angle of the water blade could be pivotable in a similar manner to the water atomising nozzles 315. A water blade may be more effective than water dispensed from nozzles for removing debris, heavy soiling, or soap from hands, and the actuation of the water blade can be incorporated into the washing processes either by user selection or automatically.

A hand dryer air outlet 430 (which may comprise multiple outlet apertures) may be provided to produce an air sheet to dry the user's hands on removal of the hands from the cavity 900.

Washing Process with Sensor Control Detail; Directional Control

The computer vision sensor unit 110 or other optical sensors situated around the display screen 100 detect when a user approaches the device and can activate the device to modify the screen contents, which may change according to user characteristics such as the approaching individual's height or other characteristics (such as sex or estimated age); the display screen options and output could also be customised according to these sensed parameters, both in terms of what options are provided, and the manner and position of where the information is placed on the display.

The device's display screen can present the user with the option of customising their wash. If the user ignores this and inserts their hands into the device's cavity, sensors 500 near the cavity entrance 910 will detect the insertion of the hands into the cavity 900. A pre-set delay affords the user enough time to adjust the position of their hands to a level that corresponds with where the liquids will start to be released from so that the user can determine how high up their hand and/or wrist they wish the wash to commence from. A warning that the water and soap application stage is about to begin may be given. The moving units will then move down the cavity, releasing water and then soap.

The sensors 500 output data to the control unit 600 and via that to the CPU 150. If the user wishes to modify this initial stage, for instance by applying extra soap or opting for a water sheet to dislodge superficial debris from the hands, they may be able to do so via gesture control from within the hand cavity or via voice command, or via the user changing the proximity of their hands from the various sensors 500, and their change of position and speed of change, in order to match the user's hand movements to a gesture in a similar way to the gesture recognition carried out by the CPU 150 on the output of the computer vision sensor unit 110.

In one embodiment sensors on the moving unit or elsewhere within the cavity will detect whether each individual nozzle's spray trajectory will make sufficient contact with the user's hands. When the user's hands are placed within the cavity, sensors within the cavity identify the location and orientation of the hands in real-time. The CPU is pre-programmed to recognise the angles at which the water atomising nozzles 315 and soap spraying nozzles 325 will disperse their respective sprays and to make adjustments to its calculations of the angle of dispersion that would occur with any variation of the pressure of the liquid flowing through the nozzles. By mapping the position of where the hands are located within the hand cavity, the CPU can determine whether the spray trajectory of each nozzle will result in sufficient water or soap contact with the user's hands. Accordingly, only those nozzles whose spray will make sufficient contact with a hand will release liquid, thus reducing needless waste of soap and water. If someone is washing only one hand, fewer nozzles could be activated, saving resources. The activation and deactivation of specific nozzles can be enabled by using nozzles with automated valves which can be controlled by the CPU. The movement of the moving unit 300 itself may also be modified to optimise the application of water and/or soap to the user's hands.

In another embodiment, sensors within the cavity will enable nozzles whose spray would otherwise not make sufficient contact with the hand to automatically adjust their angle so as to ensure that the soap and water released from them makes sufficient contact with the user's hands. Each nozzle may be attached to the soap and water outlets by a pivoting coupler that is powered by an electric motor, with the nozzle's angle of pivot controlled by the CPU. The CPU calculates the direction that each nozzle will need to move based upon the location of the hands within the cavity (as determined by sensors within the cavity) in order to optimise the hands' spray coverage and reduce unnecessary excess overshoot of liquid soap and water into the cavity. It does this by calculating the projected angle of the fluid's dispersion in relation to the positioning of the nozzles relative to the hands and calculating the necessary adjustments to the nozzles' angles of direction to increase the relative quantity of liquid soap and water that will make contact with the hands. Additionally or alternatively, the nozzle could rotate or oscillate.

Although this water and soap conserving mechanism is useful in models designed to accommodate a single user at a time, in terms of resource optimisation, this is of particularly high importance in multiuser variants of this invention.

Referring to FIGS. 5 and 6, the sensors 500 within the cavity will detect when the water and soap sprays pass the end of the fingertips, and the nozzles will cease to release liquid. The moving units may then return to their initial starting position 550 (behind the lips at the top of the cavity 900 entrance) and a warning given to alert the user how much time remains for the soap rubbing stage of the washing process. The hands may be rubbed together either inside the hand cavity or above it, according to the user's preference. The duration of the rubbing stage may be able to be altered by gesture control from within the device or above the device's cavity, or by voice command. When the alert for the end of the rubbing stage is given, the user may prolong the rubbing stage by continuing to rub their hands together. When this alert is given, if the user's hands are above the cavity, they may be informed that they should place their hands at the correct level within the hand cavity, so as to receive water during the rinsing stage.

Once a sensor detects the hands are sufficiently within the hand cavity, the user is notified that the water rinse stage is about to commence and after a very short pre-set delay the moving units begin moving down the cavity whilst releasing water in order to rinse the hands. The hands can remain still whilst this takes place or be rubbed together within the cavity to aid the removal of soap.

This process facilitates a fully automated hand wash, in CDC recommended sequence, (with only plain soap and water being used: it does not require the use of antimicrobials). It is notable that there is an uninterrupted transition between the different stages of the hand wash, and that individualisation of a wash according to the individual user's hand characteristics, (such as the hand size and its location and orientation within the cavity) is enabled.

### Other Embodiments

In another embodiment, instead of the liquid dispensing nozzles being situated on a moving unit, these may be situated in a set location along the front and back walls of the hand cavity, with some or all of the nozzles being connected by pivoting couplers powered by an electric motor. This enables a nozzle's angle of pivot, directed by the CPU, to adjust where it guides the flow of liquid as it is releasing the liquid, to ensure the angle and trajectory of the liquid spray can cover the full hand surface. This will be facilitated by sensors within the cavity identifying the location of the user's hands within the cavity. This enables the hands to receive full targeted sprays, of both water and soap, even if the hands move about or change location within the cavity, without fluid being directed at places within the cavity where the hand is not located.

The nozzles will be angled downwards in order to mitigate any splashback which would be more likely to occur if the water or soap was directed horizontally at the hands.

In another embodiment, a plurality of nozzles that can release soap and water may be situated throughout the hand cavity, with only those that will result in sufficient quantity of the liquid released making contact with the hands being activated. As the nozzles are situated on both opposing (front and back) walls of the cavity, when activated they simultaneously achieve coverage of both the palmar and dorsal sides of the hands in their fluid release, as opposed to only one side as would be achieved by a faucet's release).

Blasts of water may be dispensed at different times from nozzles located at different heights, or they may be dispensed at the same time.

Drying

When the rinsing stage is finished the display screen 100 will instruct the user where to move their hands so that the hand dryer 400 will be activated. Referring particularly to FIGS. 1 and 2, in an embodiment where the hand dryer 400 is situated at the entrance of the inside of the housing of the device, the hands will be dried by moving them up and down past sensor 510 activated jets of air. This hand drying stage will not be activated until the previous washing, and rinsing stages have been completed.

When the control unit 600 has determined that the hand drying stage of the wash should commence, or that a hand drying option has been selected by the user via the touch-free display screen 100, then providing the hand drying sensor 510 can detect the presence of a hand, it will activate a motor that turns a fan 420. Referring to FIG. 8, this draws air into the device through a filter 410 which purifies the incoming air, and high velocity air then passes through the air outlets 430 onto the hands in order to dry them. The drying stage will last for a predetermined period of time which can be reduced if the hands are removed from the device meaning that they will no longer be detectable by the sensor 510 that activates the air dryer.

Once the hands are fully removed from the device, since the sensor 510 will no longer be able to detect them, the hand dryer 400 will stop, and cannot be reactivated unless an option requesting hand drying on the display screen 100 is selected and the hands are reinserted.

In an embodiment where the dryer is located on the moving soap and water units (this is not illustrated), the drying process can occur with the hands in a stationary position, without having to move them back and forth. The moving units will move up and down directing jets of air at the hands. The dryer will stop when the hands are removed from the device.

Internal Components; Washing/Drying Etc

The housing 200 includes the internal components of the handwashing device 101 Referring to FIG. 8, a control unit 600 controls the operation of the water unit 800, soap unit 810, heating unit 360, hand drying unit 400 and the moving units 300.

Water is supplied to the handwashing device 101 via a water inlet 260, where a water pump 250, main valve 265 and solenoid valve 245 directs the water to the water sheet outlets 350 and moving units 300 via a heating unit 360, second solenoid valve 365 and water flow splitters 362.

Liquid soap is stored in a soap container 210, with the liquid soap being directed to the moving unit 300 through the soap pipe 220 directed by a soap pump 230, solenoid valve 225 and soap flow splitter 285. The soap container 210 includes a soap level sensor 140.

Air is drawn from the surrounding environment through an air inlet 270 and filter 410 by a fan 420, and distributed to the hand dryer air outlets 430 via an air flow splitter 422. An air heating unit could be included if desired.

The handwashing device 101 may also include an environmental air purifier 700, having a fan 710 which draws air from the environment through an air inlet 705, through a filter 720 and ejects the filtered air through an air outlet 730.

A power supply 170 supplies electricity to the various components of the handwashing device 101.

Control Architecture

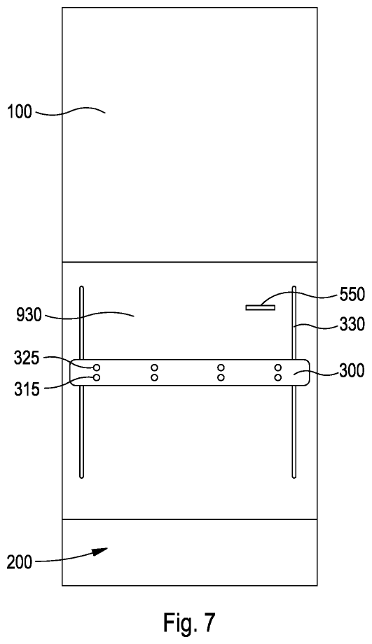

Referring to FIGS. 7 and 8, in addition to the control unit 600, the handwashing device 101 includes a CPU 150 which communicates with the control unit 600. The CPU 150 includes conventional processing components such as a logic chip and a memory. It will be appreciated that the processing and control means of the handwashing device 101 could be integrated or distributed in different ways to achieve the same functionality.

The CPU 150 is connected to the display screen 100 and computer vision sensor unit 110. A microphone 120 and speaker 125, and wireless communication module 160 are also provided, connected to the CPU 150. The CPU 150 also controls the air purifier 700.

Input Means in General

The processes of the handwashing device 101 may be controlled by several input means, ideally available and usable in combination to allow a user to utilise more than one input means, or to allow different users to utilise the input means necessary for their particular requirements.

The Input Means Ideally Comprise
(1) proximity sensors to detect the presence, location, and/or proximity of the user's hands to the sensor
(2) a microphone to detect voice commands given by the user; alternatively or additionally, the movement of a user's mouth may be detected by the computer vision sensor unit to ascertain user input (the recognition of a user's mouth movements could be useful to separate the user's instructions in a noisy environment)
(3) an interactive display screen 100 which allows the user to select options, and
(4) computer vision sensor unit 110, which ideally comprises several cameras and which can determine the position, shape, and size of a user's hands (and fingers), and by using a video capture can detect the change of motion of the user's hands and fingers; as well as using hand movement, body movement could also be utilised.

The display screen 100 and computer vision sensor unit 110 are ideally used in conjunction, so that the user can point to or move their hand or fingers near areas of the display screen 100 to select options without touching the screen. The display screen 100 could also include some proximity sensing capability, such as capacitive sensing, or less ideally touch sensitivity.

Instructions could be given by the user by means of voice commands, or by selecting options from a touch screen. Ideally though, control by detecting the position of the user's hands and fingers, and movements or gestures (ideally non-touch gestures) performed by the user, is used.

The same input means can also be used for maintenance; for example, a maintenance personnel could use voice recognition and voice control to open the handwashing device and to elicit information from it required for maintenance.

The handwashing device may be provided with the ability to process language, in order to recognise and interpret user language and in particular a user's voice commands, and in turn responding to accommodate the user's hand washing preferences.

In some embodiments there may also be a machine specific voice command displayed on the screen that deactivates or activates voice control capacity when vocalised, (e.g. a random phrase such as ‘Hey Jupiter’ or ‘Hi Coco’). An extra security safeguard could be that the device determines whether any recognised continued vocal communications sufficiently match the initial voice recognised as commanding the device. For instance, if the tone or pitch is overly altered (or the sound made sufficiently reduced) it may not recognise the command as coming from the same user even when matching the user's mouth motion with a command uttered.

By means of audio communication, the device can respond to a user's comments, by providing a wash suggestion customised according to what the device determines would be the most effective wash settings for that situation. For example, if the user states ‘my hands are really muddy’, the device might respond by suggesting ‘would you like a high pressured pre-wash?’, to which the user might reply ‘yes, but I'd like it to be with warm water’. The device can then facilitate the user's request.

This feature enables visually impaired people to be able to use the device almost as comprehensively as someone who is not visually impaired. The device may also have the ability to translate in a variety of languages, which will be useful, for instance, in an airport where many languages are spoken. Via this touch-free logic, it may also be able to respond to sign-language communication.

Gesture Control and Customisation

As previously described the handwashing device 101 includes a computer vision sensor unit 110 above the display screen 100. The computer vision sensor output is fed to the CPU 150, which can detect where the commanding hand is within the visual field of view of the camera sensors, that are located around the display screen in order to assess which area of the screen the hand is parallel to at any given point.

Most ideally, multiple camera sensors are provided, operating simultaneously to allow for the optical triangulation-based range measurement of a hand, which enables the distance of the hand from the display screen to be determined and facilitates the determination of the 3D shape of the gesture, or gestures performed by the hand as well as the hand's motion. Ideally, the multiple cameras have a wide field of vision, so that exact positioning of a user's hands is unnecessary.

The computer vision sensor unit 110 may be supplemented with infrared sensors, for the purposes of calculating depth and distance.

This computer vision sensor unit 110 feeds information back to the device's central processing unit (CPU) 150 (or a separate computer system for the display screen itself (this is not shown)) based on the user's touch-free interaction with the display screen 100, in order to extract, analyse and understand the user's touchless commands and respond in real time to them.

Based on where the hands are located in relation to the screen, and the specifics of the hand gesture made, (such as its shape, speed and trajectory), the CPU can calculate which part of the screen the user's gesture correlates with, in order to determine and facilitate the user's desired selection.

To enable this, the gesture made must be matched with a pre-set gesture (stored in a database on the device's memory) that is capable of modifying the specific wash option displayed on the area of the screen which the user's gesture is identified as being directed at.

Some wash modifications can be triggered by gestures that do not need to be made in any specific location in relation to where information is displayed on the screen. An example may be the dynamic gesture of undulating fingers in order to select a spray of water from the atomising nozzles, which may be interpreted as selecting that option regardless of where, above the hand cavity, that gesture is performed. However, other modifications to the hand wash may require that a non-touch gesture is performed in a specific location in relation to where controllable information is presented on the display screen. Alternatively, a modification request may require the trajectory of the hand to be moving towards a specific location in 3D space in relation to where controllable information is displayed on the screen. For instance, gesturing the hands in the general direction of a water temperature or water pressure icon on the display screen, may be required to initiate an increase or decrease in water temperature or pressure.

Enabling selections and/or modifications to be made on the basis of the trajectory of a dynamic hand gesture allows the option of having customisable options spread out in a clear and distinctly located manner on a display screen, which the user can easily control even if the size of the display screen (or stature of the user) means that such customisable options are displayed in locations on the screen beyond the user's natural reach.

The computer vision sensor unit 110 evaluates the scene within their field of view in order to detect and verify the presence of a user and their hands. A delay period may be included to account for a user to position their hands. If a user is detected in a front on position to the device and a sufficiently large hand is detected by the sensor, the CPU may also analyse hand characteristics such as shape, size and the direction or speed of movement it makes.

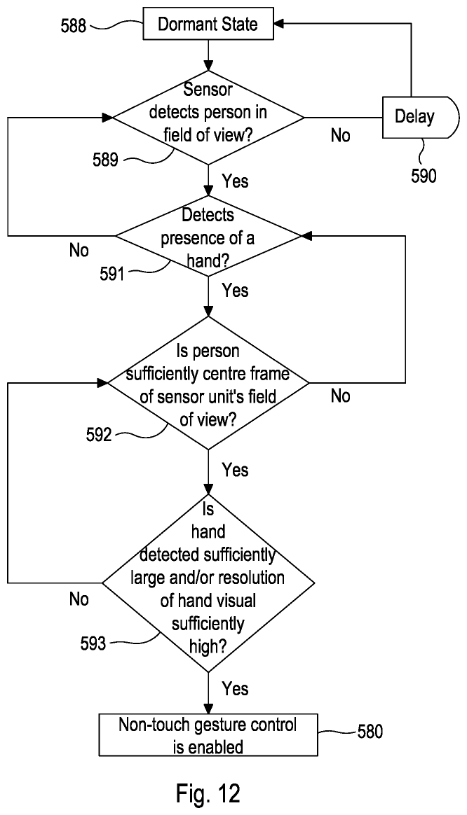

Referring to FIG. 12, the handwashing device 101 will typically be in a dormant or quiescent state 588, periodically monitoring for the presence of a user 589, 590 but not carrying out further operations or processing. When the presence of a user is detected, the handwashing device 101 will then check 591 for the presence of a hand, and if a hand is present it will check whether the user is sufficiently centred in the field of view 592, and that the resolution of the hand and/or user is sufficient for further analysis of the hand and/or user to be carried out 593. If these criteria are fulfilled, non-touch gesture control of the handwashing device 101 is enabled 580. The further steps of the gesture control process are described below in the gesture control specifics section.

It should be noted, however, that no gesture control need be required in order to automatically facilitate a full hand wash in accordance with the CDC's recommended guidelines.

The computer vision sensor unit 110 or other sensors could be used to determine the level of contamination on a user's hands automatically. For example, if a user has a really muddy section of their hands, as a pre-wash option they would probably opt for the sheet/blade of water which they can move the muddy area of their hands up and down against for however long is required to remove the excess mud.

Gesture control in general herein refers to dynamic, non-touch gesture control performed in 3D space. Dynamic non-touch gesture control requires a free flow of hand motion, an absence of physical obtrusions extends the natural area within which dynamic non-touch gesture control can be performed.

The absence of faucet, and dispensation of water and soap (and possible drying) from within its cavity allow dynamic non-touch gesture control to be more freely performed both within and above the cavity.

The absence of a faucet also allows a single camera unit located above the cavity to detect a dynamic, non-touch gesture being performed by a user anywhere in the space directly above that cavity.

This is particularly significant when using the device in conjunction with a display screen since it allows the trajectory of any dynamic gesture to be more easily determined, as there are no potential disruptions to the hands' movements, or to the field of view of any optical sensor tracking the hands' movements (in the area above the cavity itself), thus allowing the user to control any part of a screen, without potential disruption, even if the size of the display screen (or stature of the user) means that customisable options are displayed in locations on the screen beyond the user's natural reach.

Precision-targeted spraying functionality within the cavity is made possible by the detection in real-time of hand orientation, and the shape and location of each of a user's hands within the cavity. An added advantage is that this same process allows dynamic, non-touch gesture control to be performed within the cavity itself, without the need for additional sensors to be integrated within the cavity to perform this functionality.

The combination of the lack of faucet, and the presence of sensors that determine the location, orientation and shape of hands within the cavity enables non-touch gesture control to be performed within the entire space, within and above the device's cavity, in a seamless manner.

Customisation

The gesture control input means or other input means enables the user to have a great degree of control, based on their specific needs, by giving them the option to choose between different modes of wash, and to customise the various stages of the wash according to their needs. This device allows the user a varied selection of operations, and even to override or alter an initial process—for example more soap can be released, if desired, after the initial soap release has already been activated. Any such changes can be done by touch-free interaction with the display screen.

However dynamic gesture control can also be performed without the use of a display screen and can be done both from within and above the cavity in a manner that does not interrupt the natural flow of the wash process. For example, the effect of undulating fingers from deep within the cavity may activate additional water rinsing (without interrupting the flow of the wash process), or continuing to rub the hands together during the rubbing stage may prolong the actual rubbing stage by delaying the release of water onto where the hands are located.

However, there can also be the option to control the device by touching the display screen—for instance, if maintenance personnel want to type in a passcode on the screen in order to unlock the machine to replace a filter or cleansing fluid, or for other maintenance purposes. People currently do not have the ability to extensively control their mode of hand washing and have an interactive hand washing experience in a touch-free manner at the same time. This multi-functional washing device allows the user far more control, and more efficient washing than current systems permit.

Examples of washing selections which can enable greater control include, (but are not restricted to):
A pre-wash for removing excess material from the hands, (for example if there is a lot of mud or sticky food), or for when hands have been in particularly offensive environments. This allows for a rapid high pressure wash and rinse before the standard wash, or another selected wash, begins.
A standard wash which is the default washing sequence designed to follow standard recommended washing guidelines, unless the owner decides to modify this.
A heavy duty wash for particularly dirty or contaminated hands. A higher water pressure may be used, and for a longer period of time.
A quick wash which is aimed for situations where a standard wash is not appropriate (for example, a small amount of fruit juice on the hands), or where the user simply wants a quick freshen up rather than a full wash. It can also be selected when a user is in a hurry and would not wash their hands at all if doing so entailed a longer process.
A rinse only option, which would prove beneficial for some people who have bad eczema and wash their hands with emollients instead of soap—people with this problem sometimes carry their own solution around. They can rub their own moisturiser onto their hands and then opt for a rinse only mode. Unlike conventional taps, which also facilitate this ability, this water rinse will, by virtue of the device's design, cover the whole of the hands.

As previously discussed, the application of the water, and the liquid soap, can be customised both by user input, and by sensed data such as the location of the user's hand within the cavity. The handwashing device 101 system ideally uses sensors to automatically modify the volume and targeted location of the cleansing fluid and water that it releases depending on the position and size of the users' hands. The water spray can be atomised. These factors will significantly reduce the amount of water needed to wash hands, especially when compared with the amount of water that is unnecessarily wasted when standard taps are used. The quick wash option, used to freshen up hands, (for example, getting rid of a little sticky fruit juice), has an even greater impact on water conservation. This mitigates avoidance of washing due to the process being unnecessarily time-consuming. With water being released from atomising nozzles onto both sides of the hands simultaneously it can also be more resource efficient than the equivalent action achievable by a running faucet.

In addition to selecting various modes of wash at the start of the washing process, there may also be an option to activate extra soap, extra rinse or a longer hand rubbing time during the hand washing process.

This device addresses a limitation with pre-existing systems in that current automatic hand washing devices only allow a very limited choice, if any, over the washing process. Conversely, this device enables a wide range of choices based on the user's actual needs.

Multiple User

There may also be an option of an extended touch-free hand washing device designed to accommodate multiple users at the same time, when an elongated, multi-user handwashing device is provided.

Referring to FIG. 14, the extended washing device has a similar shape and design to the single cavity device but its horizontal width from left to right will be notably longer, and its appearance is akin to an elongated trough. Users will have even greater freedom of hand movement. Although there is a long line of nozzles that actively target-spray fluid onto the hands, only those nozzles that have the hands in their target range will be activated to dispense the soap and water. Accordingly, the user can activate release of fluid from anywhere along the horizontal width of the device, which can customise the wash according to the user's individual hand locations.

This is in contrast with conventional multi user washing stations, where multiple faucets share an elongated sink and both the location the user stands in and where they place their hands are largely dictated by the positioning of the faucets. An additional advantage of the invention disclosed in this specification is that the elongated device can accommodate a larger number of people washing their hands simultaneously than would be possible in a extended sink (of equal horizontal length) with multiple faucets.

This is not only space-saving, but it also reduces the number of control boards and other fittings such as pumps, water pipes, air pipes and filters that are needed. Additionally, it optimises the number of users that can wash their hands at the same time, rather than the conventional logic of one user per basin, meaning reduced time spent waiting to wash the hands. This can be with or without a display screen, although the inclusion of a touch-free screen is optimal for encouraging greater engagement in the hand washing process and allowing more personalised user control.

As well as the type or sequence of wash, the handwashing device could provide a means for the user to input or alter preferred water, soap and drying temperatures and pressures.

The handwashing device 101 may also allow the user to alter the washing process as the washing process is underway; this is more efficient both for the user's time and the handwashing resource usage than having a user simply restarting or repeating the washing process. This alteration during the handwashing process may be accomplished by the user employing some of the previous listed input types, such as voice control, or by using hand gestures within the cavity.

Gesture Control Specifics

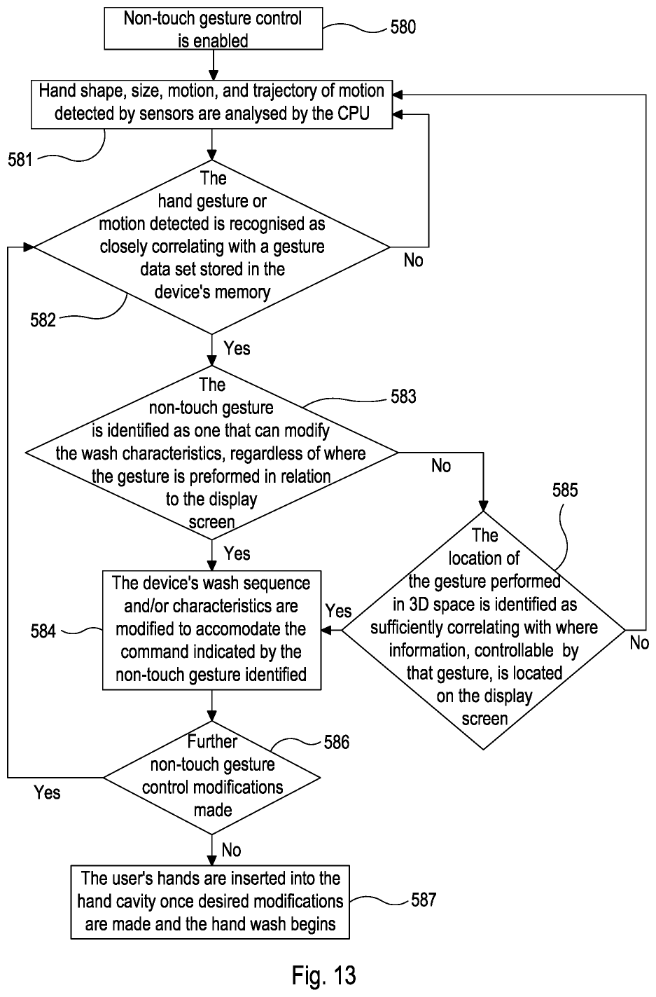

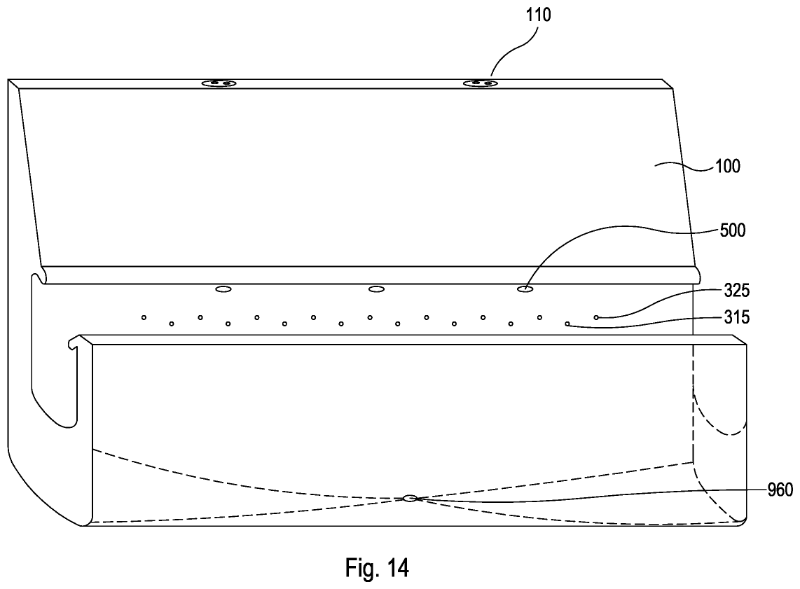

Referring to FIG. 13, when non-touch gesture control is enabled 580, the CPU 150 determines 581 the hand shape, size, motion, and trajectory from the data input from the computer vision sensor unit 110.

When there is a sufficiently close match between a detected 582 non-touch hand gesture and a pre-set digital model stored by the CPU, the hand gesture and its location in 3D space are marked digitally.

The CPU correlates the visual detected (i.e. the hand gestures made and their location in 3D space) with the location of information displayed on the screen at the time when the hand gesture is made.

If the detected gesture sufficiently matches a gesture stored in the device's database that can modify a specific hand wash option that the user's hand is identified 583, 585 as gesturing in relation to, then the selection and/or alteration of wash sequence that can be triggered by that gesture is activated 584.

The computer vision sensor unit 110 continues to monitor 586 the characteristics of the user's hands, to detect further gestures which may indicate a modification of the user instructions, essentially repeating the previous steps. When the user has conveyed their instructions to the handwashing device 101, their hands are inserted into the cavity 900 for the washing and drying steps to be performed. Alternatively, if the gesture control is performed within the cavity the hand washing may be modified even more seamlessly without the user needing to insert their hands into the cavity again to continue the washing process.

The gesture control recognition could be trained using the procedure proposed in ‘Human hand gesture recognition using a convolution neural network’ by H. Lin, M. Hsu and W. Chen, 2014 IEEE International Conference on Automation Science and Engineering (CASE), Taipei, 2014, pp. 1038-1043, doi: 10.1109/CoASE.2014.6899454. The resulting algorithm is then uploaded and implemented to the CPU of the handwashing device 101.

Artificial intelligence or other automated reasoning and logic could be incorporated in the handwashing device 101 in order to model, store, analyse and predict information based upon data the device ascertains from various users and the device's memory store in order to learn from a broad base of user habits and their preferred washing settings and choices without requiring direct knowledge of each specific user's needs or their direct active input, and with this learned behaviour responding automatically over time to user preference.

Display Screen Aspects

As discussed herein, the computer vision sensor unit 110 can be used to determine a user's interaction with the display screen, allowing the user to interact with the display screen 100 in a touch-free manner, in order to extract, analyse and understand the user's touchless commands and respond in real time to that.

In this way the display screen 100 can not only be used to control the type of hand wash desired by the user, according to their specific touch-free selection, but can also further enhance the hand washing experience by providing the user with an immersive interactive control over what is displayed on the screen 100, providing the hand washing process with a gaming like reward, without the detriments of gaming. The logic of this is that the user is more likely to proactively engage with the hand washing experience and for longer. This immersive experience is enhanced by the lack of need for a faucet in the space above the cavity.

In addition to the touch-free nature of the display screen 100, there can be the option for the screen to also be capable of responding to user touch. This combination enables the user to wash their hands using their preferred mode of control, whether initiating this by touch or in a touch-free manner—whilst a touch-free approach may be more desirable for a typical user for hygiene reasons, the ability for maintenance personnel to, for instance, quickly access the device (to replace soap or a filter) via typing a passcode onto a touch-responsive display screen to grant access to it, would likely be preferable.

In addition to this the display screen 100 has the functionality of allowing the user to interact in a touch-free manner with content, (selected or even created by the owner or maintenance personnel themselves), which can be remotely uploaded to it. This may be in the form of videos, text, images, and advertisements or any combination of these. To enhance the user experience and ease of control there is the option for incorporating a speaker and microphone into the device, and these could conveniently be located around the display screen 100.

There is an option of having a smart interactive touch-free mirror 105 (shown in FIG. 8) as an alternative to the standard touch-free display screen 100, which can operate in the same manner as the standard touch-free display screen, with the added benefits that accompany having a mirror. These benefits are not only associated with the ability of an individual to use a mirror whilst washing their hands, which is something people are often accustomed to doing, but also the novelty this would bring to an automated touch-free hand washing device in terms of the degree of control and customisability exerted by the user. The fact that there is no need for a faucet (to enable effective handwashing to be achieved) gives even greater visibility and capacity for non-touch gesture control to be performed above the cavity. The lack of a physical object between the user and the mirror makes the mirror more aesthetically pleasing.

The small, enclosed and relatively restricting single-hand cavities that typify automatic sanitising devices that use antimicrobials, and the presence of a faucet that protrudes over a sink both pose substantially increased risks of a user's hands accidentally making direct contact with a physical surface if the user is looking at information that is presented directly in front of where they are standing whilst receiving fluid onto their hands. Furthermore, with antimicrobial releasing devices, in order to consistently ensure an effective handwash, they would still require an additional faucet above their cavity spaces, in particular when dealing with heavy debris.

In addition, it has a psychological and aesthetic appeal, particularly since hand washing devices are often situated in small rooms without windows, and because mirrors have the effect of making a smaller room appear more spacious and light.

The display screen can present information in video, image or text format. A touch-free operation of the display screen adheres to the logic of reducing the risk of contamination and the spread of infections, whilst enabling greater scope of functionality and enhancing the device's appeal. In a touch-free manner, the user can select a number of wash options and actively interact with advertisements, messages, images and/or videos. The option of incorporating a microphone and speaker into the device adds an additional layering to communicating with it in a touch-free manner.

The display screen 100 can show educational, informative, instructive or advertising material with the opportunity for the user to actively interact with these displayed features without increasing the risk of hand contamination due to the touch-free control afforded to the user. This also adds to the inherent intrigue of the device to engage the user, and means that the user is less likely to become bored whilst washing their hands, and is more likely to engage effectively in the hand washing process.

There is the ability for this breadth of possible information displayed on the device to be remotely updated, by wireless means. For example, if new hand washing guidelines in the event of a change of medical guidance arises, the owner or maintainer of the handwashing device (or the building it resides in) can upload specific guidance and instructional videos.

The device's control unit can also be used to analyse and provide remote feedback to the owner or maintenance user on the device's usage and maintenance requirements, so that they can be alerted to matters such as when soap or a filter might need replacing, or servicing is required. This data analysis and feedback can allow for a more streamlined running of the machine.

The display screen 100 may provide a visual display on the screen that transforms while the user rubs soap off their hands, according to how they are moving their hands. This is enabled by the computer vision sensor unit 110 on the device. Slight variants of hand movement can lead to different visual effects, meaning that the hand washing experience verges on creating an immersive interactive effect that not only engages the user to wash their hands, but encourages them to wash their hands for longer in a more effective manner, and gives a psychological reward for correct hand washing. Similarly, the display screen 100 images may transform as the user rubs soap off their hands according to the user's hand movements.

The primary aim of the handwashing system is to encourage the users to perform sufficient and appropriate hand washing in accordance with the CDC's recommended handwashing guidelines and to do so in a resource-efficient manner. The purpose is not to try to enforce compliance by surveillance monitoring tactics that could result in punitive measures taken against those who do not comply with a specific protocol, but rather to create a new device and system that functions in a way that automatically ensures a user's adherence to the correct CDC recommended sequence, using plain soap and water.

Of note, many current automatic hand washers that seek to enforce better hand washing have an in-built system for monitoring a user's compliance with the device. My innovation is not attempting to enforce hand washing by control management, but is designed to facilitate the improvement of hand washing by engaging the user, providing them with the psychological reward of directly controlling and effectively altering the nature of the content that appears on the screen in real-time, during the washing process itself, thus giving the hand washing experience a dopamine inducing gaming-like effect. A novel strand of this control is the ability of the user to modify, in a touch-free manner, their selected wash during the wash process.

By being able to perform gesture control within the cavity, the user does not have to move their hands from where gesture control is performed in order to continue their hand wash procedure from within the cavity. If the modification is done mid-wash then gesture control outside the cavity could pose the problem of dripping wet hands, with contaminated water and soap potentially falling onto surrounding surfaces (including the floor) thus spreading germs.

The content displayed on the display screen is ideally customised both for the particular wash process selected by the user, and the detected characteristics of the user. For example, the directions and information provided to a user with muddy hands can be different to that provided for a user with relatively clean hands.

The content to be displayed on the display screen (and indeed types of wash process that are available), can be updated via the wireless communication module 160.

Rather than providing a display screen directly attached to the handwashing device 101, a separate display screen could be provided, allowing the display screen to be placed at a different height, and to electrically isolate it from the washing and drying machinery of the handwashing device.

Processes

As described herein, aspects of the handwashing device 101 may be controlled by interactive instructions given by the user, some aspects though may be controlled principally by the use of proximity sensors in the cavity 900.

When the sensor 510 first detects the presence of a hand in the hand washing device then, unless an alternative option such as drying has been selected via the display screen 100, the hand washing process will begin. The control unit 600, after a pre-set/determined delay which enables the user to position their hands properly within the device, will activate the solenoid valve 245 to allow water, pressurised with the aid of a pump 250, to flow through the water outlets 310—this water may be atomised via an atomising nozzle 315.

The control unit 600, also activates the solenoid valve 225 to allow liquid soap, pressurised with the aid of a pump 230, to flow through the soap flow splitter 285 and soap outlets 320. This is done simultaneously with the water dispensation, or slightly before or after.

The hand rubbing stage then begins; a countdown and visual instructions of correct hand rubbing technique may appear on the display screen 100.

The user has the option of either rubbing their hands together within the machine 900 or outside of it. If the user rubs their hands together outside of the machine, at this point during the hand washing procedure, a computer vision sensor unit 110 detecting this hand rubbing motion can trigger an interactive visual (that reacts to the user's hand motions) to appear on the display screen 100, thus rewarding the user psychologically for taking sufficient time to rub their hands.

The display screen 100 may also show instructions of the hand washing method, including a way that enables the user to override the standard time sequence afforded to hand rubbing and allow the rinsing stage to be brought forward.

This ability to override the countdown will be set by the control unit 600 to only be allowed to occur after a set period of time, and will be established by placing the hands in their original starting position within the housing unit 900 and keeping them relatively still (i.e. not actively rubbing them). This relative stillness will be detected by a sensor 500 which will relay this to the control unit 600, and set the rinsing stage in motion.

Alternatively, the user can continue rubbing their hands together within the device 900 until the countdown ends, when they will be instructed via the display screen 100 to return their hands to the original starting position. If the user needs more rubbing time, they can keep rubbing their hands together within the device. There will be an upper time limit after which, no matter the positioning or motion of the hands, providing they are still within the unit, the water rinsing stage will begin regardless.

Shortly before the rinsing stage does commence, a visual (and possibly audible) alert on the display screen 100 will notify the user that the rinse stage is about to begin.

The control unit 600 will trigger the moving units 300 to move downwards away from their resting place in the direction of the fingertips via the motorised pulley/trolley system 330 and associated motor 290. At the same time, with the aid of a water pump 250 and a solenoid valve 245, the control unit 600 will initiate the release of pressurised water through the water outlets 310 on the moving units 300. Sensors 500, meanwhile, will encourage the water to target the hands to ensure their full coverage whilst limiting water wastage.

When the water passes the fingertips, the moving units 300 once again move up the hands, to the ‘level’ 550 at which the washing process started, still releasing water. At any point during the rinsing stage the hands can be rubbed together within the device, if desired, to improve the soap removal. The process is continued with the final rinse being completed when the water spray passes the ends of the fingers and the water spray stops.

Further rinses, with the moving units 300 travelling up and down the hand, may be enabled by either selecting this option via the display screen 100 at the start when the mode of wash is selected, (e.g. with an extra rinse mode), or by activating a longer rinse on the display screen 100 during the rinse itself. There can also be an option for increasing the water pressure to aid more thorough rinsing.

The moving units 300 and/or the water blades could be utilised to sterilise the cavity when not in use.

Rapid Rinse Selection

When a long wash time is given as a default, this may result in people simply avoiding washing when there is only a requirement for a rapid rinse, and likewise, if a person is in a hurry, hand washing may be avoided if the process is too time consuming. The described system therefore allows people to select a light and rapid rinse when appropriate, for example if fruit juice has made the hand sticky, such that a very limited and pre-automated option would otherwise mean that they must go through an unnecessarily long, and also wasteful, washing process.

It also encourages people to wash their hands who would otherwise be disinclined to wash their hands if doing so meant they have to touch a surface (such as a faucet) or control mechanism which others may have touched with dirty hands.

There is a need to motivate more frequent and more efficient hand washing practices. This device meets the need to inspire greater adoption of hand washing by developing user confidence in the effectiveness of the hand washing method and by avoiding time wasted.

The rapid initial water, followed by soap application, the seamless and automated nature of the wash, and the fact that there is full hand coverage on both sides of the hands simultaneously during the soap and water application stages (with these applications being visibly clear to the user given the sizable open nature of the cavity) means that it is quicker to perform the recommended CDC hand wash in an effective manner than is possible with a sink and faucet. It also provides the user with greater confidence of an effective hand wash having been achieved, particularly in light of the wide capacity for errors and human variation that occurs during the handwashing process using a sink and faucet. The soap-glove effect ensures that there is no user doubt as to whether sufficient soap has been used during the handwashing process.

User engagement is further enhanced by virtue of the instructive, informative, educational and advertising material displayed on the screen that the user can interact with in a touch-free manner, and by creating different modes of wash that the user can easily choose between in a touch-free manner.

Soap Levels; Filters

Hand washing locations need soap replacement, and those with hand dryers need filter replacements. Wherever the hand washing location involves a device integrating electronics, servicing and maintenance are also important considerations. The ability to effectively wash hands is prevented when such devices are not adequately maintained, and most commonly when they run out of soap. Without advanced notice that resources are nearly depleted being provided to those maintaining the device, and without remaining levels of, for instance, cleansing liquid being clearly relayed to the user, the problems relating to this are exacerbated. The CPU 150 monitors the soap level sensor 140 and transmits an alert to a building maintenance system when the soap level reaches a set low level. Similarly, the length of time that the filter 410 has been used can be monitored, and an alert transmitted when a set duration has been exceeded. These alerts may conveniently be transmitted using the wireless communication module 160.

As well as helping prevent the handwashing device 101 running out of soap, in the event that the soap has been exhausted, the handwashing device 101 can indicate this to the user via the display screen 100.

Customisation

The availability of interaction with the display screen 100 can be used to control the type of hand wash desired by the user, according to their specific touch-free selection. It can also further enhance the hand washing experience by providing the user with an immersive interactive control over what is displayed on the screen 100, providing the hand washing process with a gaming like reward. This encourages the user to engage with the hand washing experience and for longer.

The scope of the customisability that is available without the user having to physically touch the device, and which is greatly enhanced by its interactive touch-free display screen, means that this device can address the user's specific needs. Thus, the specifics of the wash may be customised according to the size of the hands and how much of the hand and arm need washing, as well as the mode of washing desired.

Transparent Front

A casing design option allows the user to view their hands through a transparent section on the front-facing wall of the device. In addition to the aesthetic appeal of this, this option is designed to increase user confidence in the hand washing process, and thereby encourage them to use the device. Further, a design may be included on the rear wall 930, for example a pair of hands, to indicate to a user where their hands are to be placed, and the transparent front allows such a design to be easily seen.

Illumination may also be provided for the cavity, preferably integrated with the transparent front. The illumination can serve to direct a user to the handwashing device in dimly lit environments, and can also be co-ordinated with the washing process and display screen (for example, the illumination could change colour or pulse) to indicate points in the washing cycle or for display.

The side walls 940 may also be transparent.

Environmental Purifier

Referring to FIG. 8, there is also an option to include an additional air purifying feature 700 within the washing device, for improving the air quality of the location of the device in a strategic manner.

This air purifying functionality may be connected to an air quality sensor 130, either on the device itself, or elsewhere in the room where it is located, which can send a wireless signal to the control unit 600 (and/or CPU 150) of the washing device when it detects that a certain level of air contaminants are present. The device's control unit 600 can then automatically activate a motorised fan 710 for this air purifying feature, so that air from the room is drawn into this additional air purifying section, and through its filter 720, before purified air is then circulated back into the room. When the air quality sensor determines that the location's air pollution is below a certain level it can send a signal to the control unit 600 of the washing device, which will automatically stop the motorised fan 710 if it is active, for energy saving reasons. Alternatively, if the additional air purifying feature has been active for a predetermined period of time, it can be programmed to automatically turn off. Similar to other information relating to the hand washing device, any information regarding this additional air-purifying functionality, such as when its filter should be replaced (and its frequency of use), can be relayed wirelessly back to the owner or maintainer of the device. They can also remotely control the purifying feature according to when they want it to be active, and this may be determined by them on the basis of the information they remotely receive from it. Data from the air quality sensor 130 can be transmitted by the wireless communication module 160 for analysis.

By having the option of combining an air purifier within the hand washing device (separate to the air filter used for the hand-dryer) it has the added benefit of being more space efficient than if these features were in separate devices.

It will be appreciated that the provision of a separate air purifier is optional. The need for this will vary on factors such as the size of the room and its general air quality, as well as whether there are other air purifiers in the vicinity, for instance, if a number of these hand-washing devices were in a single room.

Having this additional air purifying functionality means that the air filter for the hand dryer should last longer. In order to address the risks associated with air filters not being replaced when they should be, which would result in dirty air being used to dry the hands, and spread out across the room, smart feedback from the device can give advance notification of when a replacement is due, enabling a replacement filter to be automatically ordered and delivered in due time. It also mitigates the risk of unhygienic aerosol particles, that are present in plumes created when a toilet is flushed in a washroom, from landing on surfaces that are touched by the user, or even landing directly on the user's hands after they have just washed them.

Furthermore, the ability to remotely relay information to and from the device's control unit 600 and CPU 150, enables the non-hand drying air purifier to be turned on or off remotely, (such as in circumstances when its use is not necessary), in order to reduce energy consumption and conserve filter life.

The optional air purifying filter is separate to the hand drying filter. This means that if someone forgets to replace the filter for the hand dryer, given that the air in the room should be cleaner, the user has less of a chance of drying their hands with contaminated air.

The additional air purifier, incorporated to improve the air quality in the device's location, can be programmed to switch on and off automatically when the air contamination level is detected to be at certain levels of quality. An air quality detector would be required to determine the air pollution levels, whether integrated into the device or situated elsewhere in the room. Many bathrooms have poor air quality, and would benefit from having an additional air purifier for the room within a hand-washing device.

Dehumidifier

The device may additionally incorporate within the housing a dehumidifying section as opposed to an air purifying section, or this dehumidifying functionality may be incorporated with the air purifier as a combined air purifier and dehumidifier.

Such a dehumidifier is particularly useful in a damp room, and like the air purifier solves the issue of needing multiple devices whilst maximising spatial efficiency. Its dehumidifying effect, like the air purifying effect, can be programmed to turn on or off according to user traffic.

The air purifier and/or dehumidifier are features that can be incorporated in embodiments of the hand washing device, providing a novel method of optimising conditions in a restroom or similar setting via an all-in-one device, thereby saving space, and cutting down on electrical and water drainage points. It also means that these varied elements can simply be controlled in a programme that works in tandem with when others are near to or using the device or when there is heavy use of the room. This is particularly pertinent to diminish any impact possibly caused by the noise emanating from motorised fans. Since the additional features are contained within a single unit this makes it very simple to orchestrate and harmonise their respective functioning and can have the effect of improving the environment of the room, and thus facilitating an inviting hand-washing experience.

Washrooms, in particular, are often damp environments where moisture in the air can facilitate the growth of pathogenic spores and bacteria. Since my hand washing device is already connected to a drainage outlet it means that the addition of a dehumidifier would render it naturally suited to disposing of the moisture it removes from the air.

Ideally the dehumidifying section would be situated behind the back wall of the inner cavity, and above the drainage outlet, so that it can naturally self-drain without any complex mechanical requirements to achieve this effect.

It will be realised that the CPU 150, associated arithmetic and logic unit, and memory, and the control unit 600, could be integrated, or different control means could be distributed through the device in various ways as is well known in the art.

The system is smart in operation, with its sensor capacity capable of determining the real time location of hands in 3D space. This context awareness operates in conjunction with the cavity's integrated smart water and soap targeted release mechanisms, to optimise resource efficiency and handwashing efficacy.

The system has fully autonomous operational capacity—it performs tasks autonomously without the need for the direct command of a user, in contrast with basic handwashing appliances that need individual user activation to trigger the release of a specific fluid: e.g. placing a hand beneath, or in front of, a specific IR proximity sensor, in order to dispense water, or soap.

The system has a computer processor and sensors that enable AI functionality

The system has connectivity between sensors and the CPU, and an ability to connect wirelessly to other devices in order to facilitate its process of autonomous intelligent decision making.

The system has context awareness, whereby it can perceive a wide range of information from its surrounding environment through sensors, which can then be utilised to make autonomous decisions and to provide direct assistance to the user.

The following combinations of elements and features are of particular interest:

A001.

A smart handwashing device comprising:
a handwashing cavity large enough to allow freedom of hand movement and the rubbing of both hands together within it;
a dispenser with nozzles, for distributing water and soap, are in communication with a CPU;
sensors which are capable of determining the real time location of the hands within the cavity,
wherein the CPU uses the sensed data of the hands' location in real time to determine which nozzles should be activated to release fluid onto the hands, and/or
the direction or trajectory of the nozzles' fluid release,
such that the trajectory of fluid released makes contact with the hands based on the location of each nozzle within the cavity in relation to the detected location of the hands within the cavity;
wherein the device is further characterised by the CPU directing an automated sequence of targeted water and soap release, with the soap and water's targeted application further characterised by the lack of need for specific hand placement/location within the cavity to receive any of the separately dispensed fluids, and with the nozzles targeted application of spray being directed to both sides of both hands simultaneously.

A002 A smart handwashing device comprising:
a handwashing cavity large enough to allow freedom of hand movement and the rubbing of both hands together within it;
a dispenser with nozzles, for distributing water and soap, within the cavity, and are in communication with a gesture detection system;
sensors comprising at least one camera located above the cavity,
wherein the dispenser is capable of delivering more than one wash process (to both sides of the hands simultaneously), the particular wash process being determined by (the non-touch, dynamic) gestures of the user.
the gesture detection system capable of analysing hand movements and assigning an instruction from the hand movements,
a washing control system which accepts the assigned instructions from the gesture control system, and initiates or controls the initiation, duration and cessation of operation of water and/or soap dispensation on the basis of the assigned instructions

A003. A smart handwashing device comprising:
a handwashing cavity large enough to allow freedom of hand movement and the rubbing of both hands together within it;
a dispenser with nozzles, for distributing water and soap, wherein the nozzles are situated on parallel moving cleaning units located on opposing walls of the cavity which traverse the vertical height at least part of the cavity to release liquid soap and water onto the hands, with each moving unit being characterised by a bar whose movement is electrically powered, and within which soap and water pipes deliver fluid to dispensing nozzles on the unit;
wherein the parallel cleaning moving units are in communication with a CPU,
sensors, situated on at least two opposing walls of the cavity, which are capable of determining the real time location of the hands within the cavity,
wherein the CPU uses the sensed data of the hands' location in real time to determine which nozzles should be activated to release fluid onto the hands, and/or the direction or trajectory of the nozzles' fluid release,
such that the trajectory of fluid released makes contact with the hands based on the location of each nozzle within the cavity in relation to the detected location of the hands within the cavity;
wherein the device is further characterised by the CPU directing an automated sequence of targeted water and soap release, with the soap and water's targeted application further characterised by the lack of need for specific hand placement/location within the cavity to receive any of the separately dispensed fluids, and with the nozzles targeted application of spray being directed to both sides of both hands simultaneously.

A004. A smart handwashing device comprising:
a handwashing cavity large enough to allow freedom of hand movement and the rubbing of both hands together within it the width being elongated and capable of accommodating multiple users simultaneously
a dispenser with nozzles, for distributing water and soap, are in communication with a CPU;
sensors which are capable of determining the real time location of the hands within the cavity,
wherein the CPU uses the sensed data of the hands' location of each user in real time to determine
which nozzles should be activated to release fluid onto the hands of each user, and/or the direction or trajectory of the nozzles' fluid release onto the hands of each user, such that the trajectory of fluid released makes contact with the hands based on the location of each nozzle within the cavity in relation to the detected location of the hands within the cavity;
wherein the device is further characterised by the CPU directing an automated sequence of targeted water and soap release of each user independently, with the soap and water's targeted application further characterised by the lack of need for specific hand placement/location within the cavity to receive any of the separately dispensed fluids, and with the nozzles targeted application of spray being directed to both sides of both hands simultaneously;
wherein the initiation of each individual user's wash sequence is activated in accordance with when the individual user inserts their hands within the elongated cavity.

A005. A smart handwashing device comprising:
a handwashing cavity large enough to allow freedom of hand movement and the rubbing of both hands together within it;
a dispenser with parallel thin, elongated slots, for distributing water and soap where pressurised water is released as a water blade for shearing off debris and rinsing, are in communication with a CPU;
sensors which are capable of determining the real time location of the hands within the cavity,
wherein the CPU uses the sensed data of the hands' location in real time to determine which slots should be activated to release fluid onto the hands, and/or
the direction or trajectory of the slots' fluid release,
such that the trajectory of fluid released makes contact with the hands based on the location of each slot within the cavity in relation to the detected location of the hands within the cavity;
wherein the device is further characterised by the CPU directing an automated sequence of targeted water and soap release, with the soap and water's targeted application further characterised by the lack of need for specific hand placement/location within the cavity to receive any of the separately dispensed fluids.

A006
the slots' targeted application of spray being directed to both sides of both hands simultaneously.

A007. A handwashing device comprising

A handwashing cavity

A dispenser for water and/or cleaning fluids within the cavity
the cavity being elongated and capable of accommodating multiple users simultaneously.

A008. A smart handwashing device comprising:
a handwashing cavity large enough to allow freedom of hand movement and the rubbing of both hands together within it;
a dispenser with nozzles, for distributing water and soap, are in communication with a CPU;
sensors which are capable of determining the real time location of the hands within the cavity, a display screen for presenting information to a user;
wherein the CPU uses the sensed data of the hands' location in real time to determine which nozzles should be activated to release fluid onto the hands, and/or the direction or trajectory of the nozzles' fluid release;
such that the trajectory of fluid released makes contact with the hands based on the location of each nozzle within the cavity in relation to the detected location of the hands within the cavity;
wherein the device is further characterised by the CPU directing an automated sequence of targeted water and soap release, with the soap and water's targeted application further characterised by the lack of need for specific hand placement/location within the cavity to receive any of the separately dispensed fluids, and with the nozzles targeted application of spray being directed to both sides of both hands simultaneously;
the sensors include a microphone and/or camera capable of determining a user's mouth movements, and the processors can parse and distinguish a user's voice commands to determine, alter or modify a particular wash process in response to a user's spoken words.

A009 A smart handwashing device matching user's voice tone/level to identify that same user that initiated sequence is commanding device when further communication is made.

A0010 A smart handwashing device wherein a microphone relays a user's spoken communication to a CPU, which can parse the words spoken, and on the basis of the words spoken by the user determine whether a specific wash process should be activated, even when a user has not specifically stated the wash process they need, and initiate the determined wash process accordingly.

A0011. A handwashing device comprising
a handwashing cavity
a dispenser for water and/or cleaning fluids
a display screen
sensors and processors, capable of determining different gestures of a user
the dispenser being capable of delivering more than one wash process, the particular wash process being determined by the gestures of the user
the data from the sensors being used to generate or modify content for display on the display screen.

A0012. A handwashing device comprising
a handwashing cavity
a dispenser for water and/or cleaning fluids
sensors and processors
the dispenser being capable of delivering more than one wash process, the processors capable of storing use data, and to modify the wash process on the basis of stored use data.

A0013. A handwashing device comprising
a handwashing cavity
a dispenser for water and/or cleaning fluids
an air purifier
a display screen
sensors and processors, capable of determining the air quality of the handwashing device's environment and actuating the air purified when an air quality threshold is passed.

A0014. A handwashing device comprising
a handwashing cavity
a dispenser for water and/or cleaning fluids
a display screen including a mirrored surface.

A0015. A handwashing device according to any of the above points wherein the sensors and processors are capable of determining different gestures of a user
the dispenser being capable of delivering more than one wash process, the particular wash process being determined by the gestures of the user.

A0016. A handwashing device according to any of the above points wherein the dispenser comprises parallel moving cleaning units which traverse at least part of the cavity to release liquid soap and water onto the hands.

A0017. A handwashing device according to any of the above points wherein the sensors are capable of distinguishing the size or contamination of a user's hands, and the processors use this data to deliver a targeted wash process and/or to automatically adjust the pressure of the water or cleaning fluid applied.

A0018. A handwashing device according to any of the above points wherein the sensors and processors provided in the cavity capable of determining different gestures of a user within the cavity and altering the washing process in response to particular gestures.

A0019. A handwashing device according to any of the above points wherein the dispenser is operable to generate a water sheet.

A0020. A handwashing device to any of the above points wherein the cavity being elongated and capable of accommodating multiple users simultaneously.

A0021. A handwashing device according to any of the above points wherein the dispenser being capable of delivering more than one wash process, the sensors include a microphone, and the processors can parse and distinguish a user's voice commands to determine the particular wash process.

A0022. A handwashing device according to any of the above points wherein the dispenser is capable of delivering more than one wash process, the particular wash process being determined by the gestures of the user, and the data from the sensors being used to generate or modify content for display on the display screen.

A0023. A handwashing device according to any of the above points wherein the sensor data includes physical characteristics such as height, hand size.

A0024. A handwashing device according to any of the above points wherein the sensor data includes hand movement in cavity.

A0025. A handwashing device according to any of the above points wherein the dispenser is capable of delivering more than one wash process, the processors capable of storing use data, and to modify the wash process on the basis of stored use data.

A0026. A handwashing device according to any of the above points wherein there is included an air purifier, the sensors and processors, capable of determining the air quality of the handwashing device's environment and actuating the air purified when an air quality threshold is passed.

A0027. A handwashing device according to any of the above points wherein the display screen includes a mirrored surface.

A0028. A handwashing device according to any of the above points wherein the wash processes may be varied to include variations of length of wash, water temperature and water pressure.

A0029. A handwashing device according to any of the above points wherein the display screen is touch-sensitive, and the processors can distinguish inputs to determine the particular wash process.

A0030. A handwashing device according to any of the above points wherein the display screen may be operated in a touch-free manner, and the processors can distinguish inputs to determine the particular wash process.

A0031. A handwashing device according to any of the above points wherein the display screen is separate to the handwashing device but in communication with the processors.

A0032. A handwashing device according to any of the above points wherein the cavity includes a front wall, and side walls, the front wall extending upwards beyond the upper edge of the side walls.

A0033. A handwashing device according to any of the above points wherein the side walls are transparent.

A0034. A handwashing device according to any of the above points wherein the front wall is transparent.

A0035. A handwashing device according to any of the above points wherein the front wall is illuminated.

A0036. A handwashing device according to any of the above points wherein the processor is capable of facial or voice recognition and this is used to allow servicing, maintenance and access control to be performed.

1. A smart handwashing device comprising:
a handwashing cavity large enough to allow freedom of hand movement and the rubbing of both hands together within it;
a dispenser with nozzles, for distributing water and soap, are in communication with a CPU;
sensors which are capable of determining the real time location of the hands within the cavity,
wherein the CPU uses the sensed data of the hands' location in real time to determine which nozzles should be activated to release fluid onto the hands, and/or
the direction or trajectory of the nozzles' fluid release,
such that the trajectory of fluid released makes contact with the hands based on the location of each nozzle within the cavity in relation to the detected location of the hands within the cavity;
wherein the device is further characterised by the CPU directing an automated sequence of targeted water and soap release, with the soap and water's targeted application further characterised by the lack of need for specific hand placement/location within the cavity to receive any of the separately dispensed fluids, and with the nozzles targeted application of spray being directed to both sides of both hands simultaneously.

2. A smart handwashing device of claim 1 wherein the sensors situated on at least two opposing walls of the cavity.

3. A smart handwashing device of claim 1 wherein the nozzles are situated on at least two opposing walls within the cavity.

4. A smart handwashing device of claim 1 wherein the location of the hands within the cavity is determined by triangulation measurement of optical or lidar sensors, with these sensors relaying the sensed data to the CPU.

5. A smart handwashing device of claim 1 wherein the dispenser delivers an automated sequence of water release, and separately of soap (or water and soap) release, with the soap and water's targeted application further characterised by the lack of need for specific hand placement/location within the cavity to receive any of the separately dispensed fluids, and with the nozzles targeted application of spray being directed to both sides of both hands simultaneously.

6. A smart handwashing device of claim 1 wherein the sides of the cavity are at least partially open sides.

7. A smart handwashing device of claim 1 wherein the dispenser is capable of delivering more than one wash process, the processors capable of storing use data, and to modify the wash process on the basis of stored use data.

8. A smart handwashing device of claim 1 wherein there is included a gesture detection system capable of analysing hand movements and assigning an instruction from the hand movements,
a washing control system which accepts the assigned instructions from the gesture control system, and initiates or controls the initiation, duration and cessation of operation of water and/or soap dispensation on the basis of the assigned instructions
and the dispenser is capable of delivering more than one wash process to both sides of the hands simultaneously, the particular wash process being determined by gestures of the user.

9. A smart handwashing device of claim 1 wherein there is included a gesture database.

10. A smart handwashing device of claim 1 wherein there is included at least one camera located above the cavity.

11. A smart handwashing device of claim 1 wherein the nozzles comprise parallel thin, elongated slots, for distributing water and soap where pressurised water is released as a water blade for shearing off debris and rinsing.

12. A smart handwashing device of claim 11 wherein the slots' targeted application of spray is directed to both sides of both hands simultaneously.

13. A smart handwashing device of claim 1 wherein the sensors include a microphone and/or camera capable of determining a user's mouth movements, and the CPU can parse and distinguish a user's voice commands to determine, alter or modify a particular wash process in response to a user's spoken words.

14. A smart handwashing device of claim 13 wherein the CPU can match the user's voice tone/level to identify that same user that initiated sequence is commanding the device when further communication is made.

15. A smart handwashing device of claim 13 or 14 wherein a microphone relays a user's spoken communication to a CPU, which can parse the words spoken, and on the basis of the words spoken by the user determine whether a specific wash process should be activated, even when a user has not specifically stated the wash process they need, and initiate the determined wash process accordingly.
